(19) United States
(12) Patent Application Publication
(10) Pub. No.: US 2008/0102475 A1
(43) Pub. Date: May 1, 2008

(54) Alternatively spliced isoform of phosphodiesterase 4B (PDE4B)

(76) Inventors: Zhengyan Kan, Redwood City, CA (US); Philip W. Garrett-Engele, Seattle, WA (US); John C. Castle, Seattle, WA (US)

Correspondence Address: R. Douglas Bradley;Merck & Co., Inc., Patent Department RY60-30, P.O. Box 2000, Rahway, NJ, 07065-0907, US

(21) Appl. No.: 11/973,939

(22) Filed: Oct. 10, 2007

Related U.S. Application Data
(60) Provisional application No. 60/851,442, filed on Oct. 13, 2006.
(60) Provisional application No. 60/881,264, filed on Jan. 19, 2007.

(51) Int. Cl. C07H 21/04; C07K 16/00; C12N 15/00; G01N 33/53

(52) U.S. Cl. 435/7.1; 435/320.1; 530/350; 536/22.1; 536/23.5

(57) ABSTRACT

The present invention features nucleic acids and polypeptides encoding novel splice variant isoform of phosphodiesterase 4B (PDE4B). The polynucleotide sequence of PDE4Bsv1 is provided by SEQ ID NO: 3. The amino acid sequence of PDE4Bsv1 is provided by SEQ ID NO: 4. The present invention also provides methods for using PDE4B polynucleotides and proteins to screen for compounds that bind to PDE4B.

This application claims priority to U.S. Provisional Patent Application Ser. No. 60/851,442 filed on Oct. 13, 2006, and U.S. Provisional Patent Application Ser. No. 60/881,264 filed on Jan. 19, 2007, each of which is incorporated by reference herein in its entirety.

### BACKGROUND OF THE INVENTION

The references cited herein are not admitted to be prior art to the claimed invention.

Mammalian cyclic nucleotide phosphodiesterases (PDEs) comprise a superfamily of metallophosphohydrolases that hydrolyze cAMP or cGMP to its inactive 5′-monophosphate form. PDEs are subdivided into 11 families based on sequence homology, nucleotide specificity for cAMP and/or cGMP, and inhibitor selectivity. Additionally, most PDEs possess family-specific regulatory domains such as the Ca²⁺/calmodulin binding site (PDE1), GAF domain (PDE2), PAS domain (PDE8), and UCR domains (PDE4). PDE families contain 1 to 4 distinct subtypes encoded by different genes, from which multiple splice variants are expressed, resulting in ˜50 PDE isoenzymes that vary in tissue distribution, subcellular localization, and post-translational modifications (reviewed by Lugnier, 2006, Pharm. Ther. 109:366-398).

PDEs share a common gene structure, with a catalytic domain consisting of ˜270 amino acids; a regulatory domain between the amino terminus and catalytic domain which may contain binding sites for modulators, phosphorylation sites, phosphatidic binding sites, autoinhibitory sequences, membrane association domains, or dimerization motifs; and a domain between the catalytic domain and carboxy terminus which can be prenylated or phosphorylated by MAPKinase (reviewed by Lugnier, 2006, Pharm. Ther. 109:366-398). Between PDE families, the catalytic domain is highly conserved, with 20-45% identity. Within each family the catalytic domain sequence similarity is 75% (reviewed by Lugnier, 2006, Pharm. Ther. 109:366-398).

PDEs are critical determinants for the regulation of cellular levels of cAMP and/or cGMP. PDEs are involved in a variety of physiological functions, including vision, smooth muscle relaxation, platelet aggregation, fluid homeostasis, immune response, inflammation, and cardiac contractility (Francis et al., 2001, Prog. Nucleic Acid Res. Mol. Biol. 65:1-52).

The PDE4 family is divided into four subtypes encoded by different genes: PDE4A, PDE4B, PDE4C, and PDE4D, which all specifically hydrolyze cAMP (reviewed by Houslay et al., Drug Discov. Today 10:1503-1519). PDE4 enzymes are the closest vertebrate homologs of the dunce gene of Drosophila melanogaster, which was isolated as a mutation affecting learning and memory (Davis et al., 1989, Proc. Natl. Acad. Sci. USA 86: 3604-3608; Bolger et al., 1993, Mol. Cell. Biol. 13:6558-6571). PDE4 isoforms are mainly present in the brain, inflammatory cells, cardiovascular tissue, and smooth muscles (reviewed by Lugnier, 2006, Pharm. Ther. 109:366-398). PDE4B expression has been shown in lung, inflammatory cells, liver, and brain (reviewed in Zhang et al., 2006, Expert Opin. Ther. Targets 9:1283-1305).

PDE4 isoforms possess unique upstream conserved regions (UCRs) at their amino termini. Generally, there are three groups of PDE4 isoforms. Long PDE4 isoforms have both UCR1 and UCR2. Short PDE4 isoforms lack UCR1 (reviewed by Houslay and Adams, 2003, Biochem. J. 370:1-18). Additionally, supershort isoforms have been identified for PDE4D and PDE4A, which lack UCR1 and have a truncated UCR2 but retain functional activity (Bolger et al., 1994, Gene 149:237-244; Sullivan et al., 1998, Biochem. J. 333:693-703). To date, long and short PDE4β isoforms have been identified in humans (Bolger et al., 1993, Mol. Cell. Biol. 13:6558-6571; Huston et al., 1997, Biochem J. 328:549-558; Sheperd et al., 2003, Biochem. J. 370:429-438). PDE4B splice variants have demonstrated changes in catalytic activity and susceptibility to inhibition by rolipram (Huston et al., 1997, Biochem J. 328:549-558).

The PDE4B gene maps to human chromosome 1 (Milatovich et al. 1994, Cell Molec. Genet. 20:75-86). The Reference transcript for PDE4B, NM 002600, consists of 16 coding exons (Aceview on NCBI website accessed on Sep. 7, 2006, http:www.ncbi.nlm.nih.gov/IEB/Research/Acembly/av.cgi?c=geneid&org=9606&1=5142).

PDE4B may be modulated by a variety of mechanisms. Phosphorylation by kinases, such as PKA in UCR1 and ERK in the catalytic domain, affect PDE4B activity (MacKenzie et al., 2002, Br. J. Pharmacol. 136:421-433; Baillie et al., 2000, Br. J. Pharmacol. 131:811-819). PDE4B may also be modulated by UCR1 and UCR2. UCR1 and UCR2 may mediate both intramolecular and intermolecular interaction within and between PDE4B molecules. These interactions may be involved in regulation of PDE4B enzyme activation and sensitivity to rolipram (Beard et al., 2000, J. Biol. Chem. 275:10349-10358; Richter and Conti, 2002, J. Biol. Chem. 277:40212-40221; Richter and Conti, 2004, J. Biol. Chem. 279: 30338-30348). Additionally, DISC1, a candidate susceptibility factor for schizophrenia 1, interacts with PDE4B via UCR2. DISC1 releases PDE4B in response to elevated cAMP levels (Millar et al., 2005, Science 310:1187-1191). PDE4 subcellular distribution may be influenced by molecular interactions with binding partners. There is some evidence suggesting that UCR2 confers targeting to the perinuclear Golgi/centrosomal region by interaction with myomegalin (Verde et al., 2001, J. Biol. Chem. 276: 11189-11198). β-arrestins can also form a complex with PDE4 enzymes, providing a means for recruiting the enzyme to β2-adrenoceptors at the plasma membrane (Perry et al., 2002, Science 298:834-836).

PDE4B activity may be monitored by following the hydrolysis of the 3′ cyclic phosphate bond of cAMP as described previously (Bolger et al., 1993, Mol. Cell. Biol. 13:6558-6571; Marchmont et al., 1980, Biochem. J. 187:381-392; Shepard et al., 2004, Br. J. Pharmacology 142:339-351; Claveu et al., 2004, J. Pharmacol. Exp. Ther. 310:752-760).

PDE4B has been linked to a number of diseases and conditions. Studies of PDE4B^{-/-} mice indicate that PDE4B plays a role in neutrophil recruitment (Ariga et al., 2004, J. Immunol. 173:7531-7538) and LPS-induced signaling in leukocytes and macrophages (Jin and Conti, 2002, Proc. Natl. Acad. Sci. USA 99:7628-7633; Jin et al., 2005, J. Immunol. 175:1523-1531). Millar et al. (2005, Science 310:1187-1191) reported a balanced translocation which disrupted PDE4B in a subject with schizophrenia and a relative with chronic psychiatric illness. US2006/0088835 also describes PDE4B disruption in a patient with schizophrenia. PDE4 inhibitors are being investigated for their therapeutic value for chronic obstructive pulmonary disease (COPD) and asthma (Compton et al., 2001, Lancet 358:265-270; Rennard et al., 2006, Chest 129:-56-66; Bundschuh et al., 2001, J. Pharmacol. Exp. Ther. 297:280-290; Van Schalkwyk et al., 2005, J. Allergy Clin. Immunol. 116:292-298). Mata et al. (2005, Thorax 60:144-152) demonstrated that PDE4 inhibition is effective in decreasing EGF-induced expression of mucin gene MUC5AC in human airway epithelial cells. PDE inhibitors may also have therapeutic potential for leukemia (Ogawa et al., 2002, Blood 99:3390-3397). The anti-inflammatory effects of PDE4 inhibitors may also be useful for treating atopic dermatitis (Hanifin et al., 1996, J. Invest. Dermatol. 107:51-56). PDE4 inhibition may also be a useful therapeutic approach for defective long-term memory, Alzheimer's Disease, depression, and schizophrenia (Bourtchouladze et al., 2003, Proc. Natl. Acad. Sci. USA 2003, 100:10518-10522; Gong et al, 2004, J. Clin. Invest. 11: 1624-1634; O'Donnell and Zhang, 2004, Trends Pharmacol. 25:158-163; Maxwell et al., 2004, Neuroscience 129:101-107).

Phosphodiesterase activity can be inhibited by a number of previously identified inhibitors (reviewed in Houslay et al., 2005, Drug Discov. Today 10:1502-1519; Zhang et al., 2005, Expert Opin. Ther. Targets 9:1283-1305; Lugnier, 2006, Pharmacol. Ther. 109:366-398). PDE5 inhibitors are used for the treatment of erectile dysfunction, which include sildenafil (VIAGRA®), vardenafil (LEVITRA®), and tadalafil (CLALIS®) (reviewed by Briganti et al., World J. Urol. 23:374-384). Numerous specific inhibitors for PDE4 type enzymes, such as rolipram, roflumilast, and cilomilast, have been identified (Schwabe et al., 1976, Mol. Pharmacol. 12:900-910; Hatzelmann and Schudt, 2001, J. Pharmacol. Exp. Ther. 297:267-279; Barnette et al., 1998, J. Pharmacol. Exp.Ther. 284: 420-426). PDE4B specific compounds and antisense oligonucleotides have been disclosed (US2006/0041006; US2006/0100218; US2005/0153919). Theophylline and 3-isobutyl-1-methyl-xanthine (IBMX) are nonspecific PDE inhibitors (Nicholson et al., 1989, Br. J. Pharmacol. 97:889-897). PDE4 specific compounds with subtype selectivity have also been identified, (Claveau et al., 2004, J. Pharm. Exp. Ther. 310:752-760; Manning et al., 1999, Br. J. Pharm. 128:1393-1398).

Because of the multiple therapeutic values of drugs targeting phosphodiesterase enzymes, including PDE4B, there is a need in the art for compounds that selectively bind to isoforms of PDE4B. The present invention is directed towards a novel PDE4B isoform (PDE4Bsv1) and uses thereof.

### SUMMARY OF THE INVENTION

RT-PCR and DNA sequence analysis, and real-time quantitative PCR have been used to identify and confirm the presence of a novel splice variant of human PDE4B mRNA, PDE4Bsv1. More specifically, the present invention features polynucleotides encoding a different protein isoform of PDE4B, PDE4Bsv1. A polynucleotide sequence encoding PDE4Bsv1 is provided by SEQ ID NO:3. An amino acid sequence for PDE4Bsv1 is provided by SEQ ID NO:4.

Thus, a first aspect of the present invention describes a purified PDE4Bsv1 encoding nucleic acid. The PDE4Bsv1 encoding nucleic acid comprises SEQ ID NO: 3 or the complement thereof. Reference to the presence of one region does not indicate that another region is not present. For example, in different embodiments the inventive nucleic acid can comprise, consist, or consist essentially of an encoding nucleic acid sequence of SEQ ID NO:3.

Another aspect of the present invention describes a purified PDE4Bsv1 polypeptide that can comprise, consist or consist essentially of the amino acid sequence of SEQ ID NO:4.

Another aspect of the present invention describes PDE4Bsv1 expression vectors. In one embodiment of the invention, the inventive PDE4Bsv1 expression vector comprises a nucleotide sequence encoding a polypeptide comprising, consisting, or consisting essentially of SEQ ID NO:4, wherein the nucleotide sequence is transcriptionally coupled to an exogenous promoter.

Alternatively, the nucleotide sequence comprises, consists, or consists essentially of SEQ ID NO:3, and is transcriptionally coupled to an exogenous promoter.

Another aspect of the present invention describes recombinant cells comprising expression vectors comprising, consisting, or consisting essentially of the above-described sequences and the promoter is recognized by an RNA polymerase present in the cell. Another aspect of the present invention describes a recombinant cell made by a process comprising the step of introducing into the cell an expression vector comprising a nucleotide sequence comprising, consisting, or consisting essentially of SEQ ID NO:3, or a nucleotide sequence encoding a polypeptide comprising, consisting, or consisting essentially of an amino acid sequence of SEQ ID NO:4, wherein the nucleotide sequence is transcriptionally coupled to an exogenous promoter. The expression vector can be used to insert recombinant nucleic acid into the host genome or can exist as an autonomous piece of nucleic acid.

Another aspect of the present invention describes a method of producing PDE4Bsv1polypeptide comprising SEQ ID NO:4. The method involves the step of growing a recombinant cell containing an inventive expression vector under conditions wherein the polypeptide is expressed from the expression vector.

Another aspect of the present invention features a purified antibody preparation comprising an antibody that binds selectively to PDE4Bsv1 as compared to one or more PDE isoform polypeptides that are not PDE4Bsv1.

Another aspect of the present invention provides a method of screening for a compound that binds to PDE4Bsv1 or fragments thereof. In one embodiment, the method comprises the steps of: (a) expressing a polypeptide comprising the amino acid sequence of SEQ ID NO:4 or a fragment thereof from recombinant nucleic acid; (b) providing to said polypeptide a labeled PDE4B ligand that binds to said polypeptide and a test preparation comprising one or more test compounds; (c) and measuring the effect of said test preparation on binding of said test preparation to said polypeptide comprising SEQ ID NO:4.

In another embodiment of the method, a compound is identified that binds selectively to PDE4Bsv1 polypeptide as compared to one or more PDE isoform polypeptides that are not PDE4Bsv1. This method comprises the steps of: providing an PDE4Bsv1 polypeptide comprising SEQ ID NO:4; providing an PDE isoform polypeptide that is not PDE4Bsv1; contacting said PDE4Bsv1 polypeptide and said PDE isoform polypeptide that is not PDE4Bsv1 with a test preparation comprising one or more test compounds; and determining the binding of said test preparation to said PDE4Bsv1 polypeptide and to said PDE isoform polypeptide that is not PDE4Bsv1, wherein a test preparation that binds to said PDE4Bsv1 polypeptide but does not bind to said PDE isoform polypeptide that is not PDE4Bsv1contains a compound that selectively binds said PDE4Bsv1 polypeptide.

In another embodiment of the invention, a method is provided for screening for a compound able to bind to or interact with a PDE4Bsv1 protein or a fragment thereof comprising the steps of: expressing an PDE4Bsv1 polypeptide comprising SEQ ID NO:4 or a fragment thereof from a recombinant nucleic acid; providing to said polypeptide a labeled PDE4B ligand that binds to said polypeptide and a test preparation comprising one or more compounds; and measuring the effect of said test preparation on binding of said labeled PDE4B ligand to said polypeptide, wherein a test preparation that alters the binding of said labeled PDE4B ligand to said polypeptide contains a compound that binds to or interacts with said polypeptide.

Another aspect of the present invention provides a method of screening for a compound that binds to one or more PDE isoform polypeptides that are not PDE4Bsv1. This method comprises the steps of: providing an PDE4Bsv1 polypeptide comprising SEQ ID NO:4; providing an PDE isoform polypeptide that is not PDE4Bsv1; contacting said PDE4Bsv1 polypeptide and PDE isoform polypeptide that is not PDE4Bsv1 with a test preparation comprising one or more test compounds; and determining the binding of said test preparation to said PDE4Bsv1 polypeptide and to said PDE isoform polypeptide that is not PDE4Bsv1, wherein a test preparation that binds to said PDE isoform polypeptide that is not PDE4Bsv1 but not to said PDE4Bsv1 polypeptide contains a compound that selectively binds said PDE isoform polypeptide.

Other features and advantages of the present invention are apparent from the additional descriptions provided herein, including the different examples. The provided examples illustrate different components and methodology useful in practicing the present invention. The examples do not limit the claimed invention. Based on the present disclosure the skilled artisan can identify and employ other components and methodology useful for practicing the present invention.

### DEFINITIONS

Unless defined otherwise, all technical and scientific terms used herein have the meaning commonly understood by one of ordinary skill in the art to which this invention belongs.

As used herein, “PDE4B” refers to phosphodiesterase 4B (NP_{—}002591), also known as dunce-like phosphodiesterase E4 (DPDE4). In contrast, reference to a PDE4B isoform includes NP_{—}002591 and other polypeptide isoform variants of PDE4B.

As used herein, “PDE4Bsv1” refers to a splice variant isoform of human PDE4B protein, wherein the splice variant has the amino acid sequence set forth in SEQ ID NO:4 (for PDE4Bsv1).

As used herein, “PDE4B” refers to polynucleotides encoding PDE4B.

As used herein, “PDE4Bsv1” refers to polynucleotides that are identical to PDE4B encoding polynucleotides, except that the sequences represented by exons 1-7 of the PDE4B messenger RNA are not present in PDE4Bsv1 and are replaced with an alternative exon 1A. “Exon 1A” refers to the polynucleotides encoding the portion of intron 7 retained in PDE4Bsv1. The 3′ portion of the polynucleotide sequence of exon 1A is set forth in SEQ ID NO:2.

As used herein, “PDE4” is any isoform of any phosphodiesterase 4 from any organism, including but not limited to human phosphodiesterase 4A (PDE4A), human phosphodiesterase 4C (PDE4C), human phosphodiesterase 4D (PDE4D), and human PDE4B.

As used herein, “PDE isoform” is any isoform of any phosphodiesterase from any organism, including but not limited to human PDE1A, PDE1B, PDE1C, PDE2A, PDE3A, PDE3B, PDE4A, PDE4B, PDE4C, PDE4D, PDE5A, PDE6A, PDE6B, PDE6C, PDE7A, PDE7B, PDE8A, PDE8B, PDE9A, PDE10A, and PDE11A.

As used herein, an “isolated nucleic acid” is a nucleic acid molecule that exists in a physical form that is nonidentical to any nucleic acid molecule of identical sequence as found in nature; “isolated” does not require, although it does not prohibit, that the nucleic acid so described has itself been physically removed from its native environment. For example, a nucleic acid can be said to be “isolated” when it includes nucleotides and/or internucleoside bonds not found in nature. When instead composed of natural nucleosides in phosphodiester linkage, a nucleic acid can be said to be “isolated” when it exists at a purity not found in nature, where purity can be adjudged with respect to the presence of nucleic acids of other sequence, with respect to the presence of proteins, with respect to the presence of lipids, or with respect to the presence of any other component of a biological cell, or when the nucleic acid lacks sequence that flanks an otherwise identical sequence in an organism's genome, or when the nucleic acid possesses sequence not identically present in nature. As so defined, “isolated nucleic acid” includes nucleic acids integrated into a host cell chromosome at a heterologous site, recombinant fusions of a native fragment to a heterologous sequence, recombinant vectors present as episomes or as integrated into a host cell chromosome.

A “purified nucleic acid” represents at least 10% of the total nucleic acid present in a sample or preparation. In preferred embodiments, the purified nucleic acid represents at least about 50%, at least about 75%, or at least about 95% of the total nucleic acid in a isolated nucleic acid sample or preparation. Reference to “purified nucleic acid” does not require that the nucleic acid has undergone any purification and may include, for example, chemically synthesized nucleic acid that has not been purified.

The phrases “isolated protein”, “isolated polypeptide”, “isolated peptide” and “isolated oligopeptide” refer to a protein (or respectively to a polypeptide, peptide, or oligopeptide) that is nonidentical to any protein molecule of identical amino acid sequence as found in nature; “isolated” does not require, although it does not prohibit, that the protein so described has itself been physically removed from its native environment. For example, a protein can be said to be “isolated” when it includes amino acid analogues or derivatives not found in nature, or includes linkages other than standard peptide bonds. When instead composed entirely of natural amino acids linked by peptide bonds, a protein can be said to be “isolated” when it exists at a purity not found in nature—where purity can be adjudged with respect to the presence of proteins of other sequence, with respect to the presence of non-protein compounds, such as nucleic acids, lipids, or other components of a biological cell, or when it exists in a composition not found in nature, such as in a host cell that does not naturally express that protein.

As used herein, a “purified polypeptide” (equally, a purified protein, peptide, or oligopeptide) represents at least 10% of the total protein present in a sample or preparation, as measured on a weight basis with respect to total protein in a composition. In preferred embodiments, the purified polypeptide represents at least about 50%, at least about 75%, or at least about 95% of the total protein in a sample or preparation. A “substantially purified protein” (equally, a substantially purified polypeptide, peptide, or oligopeptide) is an isolated protein, as above described, present at a concentration of at least 70%, as measured on a weight basis with respect to total protein in a composition. Reference to “purified polypeptide” does not require that the polypeptide has undergone any purification and may include, for example, chemically synthesized polypeptide that has not been purified.

As used herein, the term “antibody” refers to a polypeptide, at least a portion of which is encoded by at least one immunoglobulin gene, or fragment thereof, and that can bind specifically to a desired target molecule. The term includes naturally-occurring forms, as well as fragments and derivatives. Fragments within the scope of the term “antibody” include those produced by digestion with various proteases, those produced by chemical cleavage and/or chemical dissociation, and those produced recombinantly, so long as the fragment remains capable of specific binding to a target molecule. Among such fragments are Fab, Fab′, Fv, F(ab)′₂, and single chain Fv (scFv) fragments. Derivatives within the scope of the term include antibodies (or fragments thereof) that have been modified in sequence, but remain capable of specific binding to a target molecule, including: interspecies chimeric and humanized antibodies; antibody fusions; heteromeric antibody complexes and antibody fusions, such as diabodies (bispecific antibodies), single-chain diabodies, and intrabodies (see, e.g., Marasco (ed.), Intracellular Antibodies: Research and Disease Applications, Springer-Verlag New York, Inc. (1998) (ISBN: 3540641513). As used herein, antibodies can be produced by any known technique, including harvest from cell culture of native B lymphocytes, harvest from culture of hybridomas, recombinant expression systems, and phage display.

As used herein, a “purified antibody preparation” is a preparation where at least 10% of the antibodies present bind to the target ligand. In preferred embodiments, antibodies binding to the target ligand represent at least about 50%, at least about 75%, or at least about 95% of the total antibodies present. Reference to “purified antibody preparation” does not require that the antibodies in the preparation have undergone any purification.

As used herein, “specific binding” refers to the ability of two molecular species concurrently present in a heterogeneous (inhomogeneous) sample to bind to one another in preference to binding to other molecular species in the sample. Typically, a specific binding interaction will discriminate over adventitious binding interactions in the reaction by at least two-fold, more typically by at least 10-fold, often at least 100-fold; when used to detect analyte, specific binding is sufficiently discriminatory when determinative of the presence of the analyte in a heterogeneous (inhomogeneous) sample. Typically, the affinity or avidity of a specific binding reaction is least about 1 μM.

The term “antisense”, as used herein, refers to a nucleic acid molecule sufficiently complementary in sequence, and sufficiently long in that complementary sequence, as to hybridize under intracellular conditions to (i) a target mRNA transcript or (ii) the genomic DNA strand complementary to that transcribed to produce the target mRNA transcript.

The term “subject”, as used herein refers to an organism and to cells or tissues derived therefrom. For example the organism may be an animal, including but not limited to animals such as cows, pigs, horses, chickens, cats, dogs, etc., and is usually a mammal, and most commonly human.

### BRIEF DESCRIPTION OF THE DRAWINGS

FIG. 1A illustrates the exon structure of human PDE4B mRNA corresponding to the known reference form of PDE4B mRNA (labeled NM_{—}002600) and the exon structure corresponding to the inventive splice variant transcript (labeled PDE4Bsv1). FIG. 1B depicts the nucleotide sequences of the exon junctions resulting from the splicing of exon 1A to exon 8 in the case of PDE4Bsv1 mRNA (SEQ ID NO:1), where the 3′ sequence of exon 1A is set forth in SEQ ID NO:2. In FIG. 1B, in the case of the PDE4Bsv1 splice junction sequence (SEQ ID NO: 1), the nucleotides shown in italics represent the 20 nucleotides at the 3′ end of exon 1A and the nucleotides shown in underline represent the 20 nucleotides at the 5′ end of exon 8.

### DETAILED DESCRIPTION OF THE INVENTION

This section presents a detailed description of the present invention and its applications. This description is by way of several exemplary illustrations, in increasing detail and specificity, of the general methods of this invention. These examples are non-limiting, and related variants that will be apparent to one of skill in the art are intended to be encompassed by the appended claims.

The present invention relates to the nucleic acid sequences encoding human PDE4Bsv1, that is an alternatively spliced isoform of PDE4B, and to the amino acid sequences encoding this protein. SEQ ID NO:3 is a polynucleotide sequence representing an exemplary open reading frame that encodes the PDE4Bsv1 protein. SEQ ID NO:4 shows the polypeptide sequence of PDE4Bsv1.

PDE4Bsv1 polynucleotide sequence encoding PDE4Bsv1 protein, as exemplified and enabled herein include a number of specific, substantial and credible utilities. For example, PDE4Bsv1encoding nucleic acids were identified in an mRNA sample obtained from a human source (see Example 1). Such nucleic acids can be used as hybridization probes to distinguish between cells that produce PDE4Bsv1 transcripts from human or non-human cells (including bacteria) that do not produce such transcripts. Similarly, antibodies specific for PDE4Bsv1 can be used to distinguish between cells that express PDE4Bsv1 from human or non-human cells (including bacteria) that do not express PDE4Bsv1.

The importance of PDE4B as a drug target for psychiatric, memory, inflammatory, and leukemia disorders including schizophrenia, depression, asthma, and COPD, is evidenced by drug studies and the presence of these phenotypes in humans and mice with mutations in PDE4B (reviewed in Menniti et al., 2006, Nat. Rev. Drug Discov. 5:660-670; Zhang et al., 2005, Expert Opin. Ther. Targets 9:1283-1305; Houslay et al., 2005, Drug Discov. Today 10:1503-1519). Given the potential importance of PDE4B activity to the therapeutic management of features of psychiatric, memory, and inflammatory disorders, it is of value to identify PDE4B isoforms and identify PDE4B-ligand compounds that are isoform specific, as well as compounds that are effective ligands for two or more different PDE4B isoforms or PDE isoforms. In particular, it may be important to identify compounds that are effective inhibitors of a specific PDE4B isoform activity, yet do not bind to or interact with a plurality of different PDE4B isoforms or PDE isoforms. Compounds that bind to or interact with multiple PDE4B isoforms may require higher drug doses to saturate multiple PDE4B-isoform binding sites and thereby result in a greater likelihood of secondary non-therapeutic side effects. Furthermore, biological effects could also be caused by the interaction of a drug with the PDE4Bsv1 isoform specifically. For the foregoing reasons, PDE4Bsv1 protein represents a useful compound binding target and has utility in the identification of new PDE4-ligands exhibiting a preferred specificity profile and having greater efficacy for their intended use.

In some embodiments, PDE4Bsv1 activity is modulated by a ligand compound to achieve one or more of the following: prevent or reduce the risk of occurrence, or recurrence of psychiatric, memory and inflammatory disorders including schizophrenia, depression, asthma, and COPD.

Compounds modulating PDE4Bsv1 include agonists, antagonists, and allosteric modulators. Inhibitors of PDE4B achieve clinical efficacy by a number of known and unknown mechanisms. While not wishing to be limited to any particular theory of therapeutic efficacy, generally, but not always, PDE4Bsv1 compounds will be used to modulate the hydrolysis of cAMP to AMP. PDE inhibitors have been used as anti-inflammatory drugs and anti-depressants (reviewed by Houslay et al., 2005, Drug Discov. Today 10:1503-1519; Zhang et al., 2005, Expert. Opin. Ther. Targets 9:1283-1305). Rolipram, roflumilast, and cilomilast, have been identified as PDE4 inhibitors (Schwabe et al., 1976, Mol. Pharmacol. 12:900-910; Hatzelmann and Schudt, 2001, J. Pharmacol. Exp. Ther. 297:267-279; Barnette et al., 1998, J. Pharmacol. Exp.Ther. 284: 420-426). PDE4 specific compounds with subtype selectivity have also been identified, (Claveau et al., 2004, J. Pharm. Exp. Ther. 310:752-760; Manning et al., 1999, Br. J. Pharm. 128:1393-1398). Therefore, agents that modulate PDE4B activity may be used to achieve a therapeutic benefit for any disease or condition due to, or exacerbated by, PDE4B activity.

PDE4Bsv1 activity can also be affected by modulating the cellular abundance of transcripts encoding PDE4Bsv1. Compounds modulating the abundance of transcripts encoding PDE4Bsv1 include a cloned polynucleotide encoding PDE4Bsv1, that can express PDE4Bsv1 in vivo, antisense nucleic acids targeted to PDE4Bsv1 transcripts, enzymatic nucleic acids, such as ribozymes, and RNAi nucleic acids, such as shRNAs or siRNAs, targeted to PDE4Bsv1 transcripts.

In some embodiments, PDE4Bsv1 activity is modulated to achieve a therapeutic effect upon diseases in which regulation of PDE4B is desirable. For example, psychiatric, memory, and inflammatory disorders such as schizophrenia, depression, asthma, and COPD may be treated by modulating PDE4Bsv1 activity.

PDE4Bsv1 NUCLEIC ACIDS

PDE4Bsv1 nucleic acids contain regions that encode for polypeptides comprising, consisting, or consisting essentially of SEQ ID NO: 4. The PDE4Bsv1 nucleic acids have a variety of uses, such as use as a hybridization probe or PCR primer to identify the presence of PDE4Bsv1; use as a hybridization probe or PCR primer to identify nucleic acids encoding for proteins related to PDE4Bsv1; and/or use for recombinant expression of PDE4Bsv1. In particular, PDE4Bsv1 polynucleotides have replaced the polynucleotide region that consists of exons 1-7 of the PDE4B gene with an alternative exon 1A (SEQ ID NO:2).

Regions in PDE4Bsv1 nucleic acid that do not encode for PDE4Bsv1, or are not found in SEQ ID NO:3, if present, are preferably chosen to achieve a particular purpose. Examples of additional regions that can be used to achieve a particular purpose include: a stop codon that is effective at protein synthesis termination; capture regions that can be used as part of an ELISA sandwich assay; reporter regions that can be probed to indicate the presence of the nucleic acid; expression vector regions; and regions encoding for other polypeptides.

The guidance provided in the present application can be used to obtain the nucleic acid sequence encoding PDE4Bsv1 related proteins from different sources. Obtaining nucleic acids encoding PDE4Bsv1 related proteins from different sources is facilitated by using sets of degenerative probes and primers and the proper selection of hybridization conditions. Sets of degenerative probes and primers are produced taking into account the degeneracy of the genetic code. Adjusting hybridization conditions is useful for controlling probe or primer specificity to allow for hybridization to nucleic acids having similar sequences.

Techniques employed for hybridization detection and PCR cloning are well known in the art. Nucleic acid detection techniques are described, for example, in Sambrook, et al., in Molecular Cloning, A Laboratory Manual, 2^{nd} Edition, Cold Spring Harbor Laboratory Press, 1989. PCR cloning techniques are described, for example, in White, Methods in Molecular Cloning, volume 67, Humana Press, 1997.

PDE4Bsv1 probes and primers can be used to screen nucleic acid libraries containing, for example, cDNA. Such libraries are commercially available, and can be produced using techniques such as those described in Ausubel, Current Protocols in Molecular Biology, John Wiley, 1987-1998.

Starting with a particular amino acid sequence and the known degeneracy of the genetic code, a large number of different encoding nucleic acid sequences can be obtained. The degeneracy of the genetic code arises because almost all amino acids are encoded for by different combinations of nucleotide triplets or “codons”. The translation of a particular codon into a particular amino acid is well known in the art (see, e.g., Lewin GENES IV, p. 119, Oxford University Press, 1990). Amino acids are encoded for by codons as follows:

A=Ala=Alanine: codons GCA, GCC, GCG, GCU

C=Cys=Cysteine: codons UGC, UGU

D=Asp=Aspartic acid: codons GAC, GAU

E=Glu=Glutamic acid: codons GAA, GAG

F=Phe=Phenylalanine: codons UUC, UUU

G=Gly=Glycine: codons GGA, GGC, GGG, GGU

H=His=Histidine: codons CAC, CAU

I=Ile=lsoleucine: codons AUA, AUC, AUU

K=Lys=Lysine: codons AAA, AAG

L=Leu=Leucine: codons UUA, UUG, CUA, CUC, CUG, CUU

M=Met=Methionine: codon AUG

N=Asn=Asparagine: codons AAC, AAU

P=Pro=Proline: codons CCA, CCC, CCG, CCU

Q=Gln=Glutamine: codons CAA, CAG

R=Arg=Arginine: codons AGA, AGG, CGA, CGC, CGG, CGU

S=Ser=Serine: codons AGC, AGU, UCA, UCC, UCG, UCU

T=Thr=Threonine: codons ACA, ACC, ACG, ACU

V=Val=Valine: codons GUA, GUC, GUG, GUU

W=Trp=Tryptophan: codon UGG

Y=Tyr=Tyrosine: codons UAC, UAU

Nucleic acid having a desired sequence can be synthesized using chemical and biochemical techniques. Examples of chemical techniques are described in Ausubel, Current Protocols in Molecular Biology, John Wiley, 1987-1998, and Sambrook et al., in Molecular Cloning, A Laboratory Manual, 2^{nd} Edition, Cold Spring Harbor Laboratory Press, 1989. In addition, long polynucleotides of a specified nucleotide sequence can be ordered from commercial vendors, such as Blue Heron Biotechnology, Inc. (Bothell, Wash.).

Biochemical synthesis techniques involve the use of a nucleic acid template and appropriate enzymes such as DNA and/or RNA polymerases. Examples of such techniques include in vitro amplification techniques such as PCR and transcription based amplification, and in vivo nucleic acid replication. Examples of suitable techniques are provided by Ausubel, Current Protocols in Molecular Biology, John Wiley, 1987-1998, Sambrook et al., in Molecular Cloning, A Laboratory Manual, 2^{nd} Edition, Cold Spring Harbor Laboratory Press, 1989, and U.S. Pat. No. 5,480,784.

PDE4Bsv1 Probes

Probes for PDE4Bsv1 contain a region that can specifically hybridize to PDE4Bsv1target nucleic acids, under appropriate hybridization conditions and can distinguish PDE4Bsv1 nucleic acids from each other and from non-target nucleic acids, in particular PDE4B polynucleotides not containing exon 1A. Probes for PDE4Bsv1 can also contain nucleic acid regions that are not complementary to PDE4Bsv1 nucleic acids.

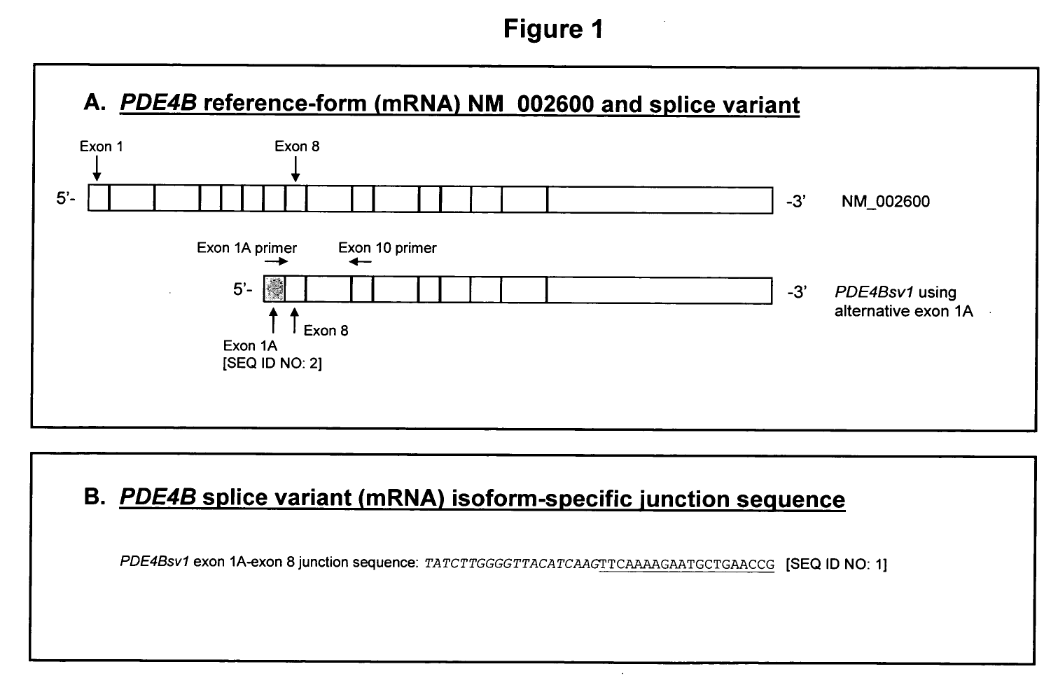

In embodiments where, for example, PDE4Bsv1 polynucleotide probes are used in hybridization assays to specifically detect the presence of PDE4Bsv1 polynucleotides in samples, the PDE4Bsv1 polynucleotides comprise at least 20 nucleotides of the PDE4Bsv1 sequence that correspond to the respective novel exon junction or novel polynucleotide regions. In particular, for detection of PDE4Bsv1, the probe comprises at least 20 nucleotides of the PDE4Bsv1 sequence that corresponds to an exon junction polynucleotide created by the alternative splicing of exon 1A to exon 8 of the primary transcript of the PDE4B gene (see FIGS. 1A and 1B). For example, the polynucleotide sequence: 5′ TTACATCAAGTTCAAAAGAA 3′ (SEQ ID NO:5) represents one embodiment of such an inventive PDE4Bsv1 polynucleotide wherein a first 10 nucleotide region is complementary and hybridizable to the 3′ end of exon 1A of the PDE4Bsv1 gene and a second 10 nucleotide region is complementary and hybridizable to the 5′ end of exon 8 of the PDE4Bsv1 gene (see FIG. 1B).

In some embodiments, the first 20 nucleotides of a PDE4Bsv1 probe comprise a first continuous region of 5 to 15 nucleotides that is complementary and hybridizable to the 3′ end of exon 1A and a second continuous region of 5 to 15 nucleotides that is complementary and hybridizable to the 5′ end of exon 8.

In other embodiments, the PDE4Bsv1 polynucleotide comprises at least 40, 60, 80 or 100 nucleotides of the PDE4Bsv1 sequence, that correspond to a junction polynucleotide region created by the alternative splicing of exon 1A to exon 8 in the case of PDE4Bsv1. The PDE4Bsv1polynucleotide is selected to comprise a first continuous region of at least 5 to 15 nucleotides that is complementary and hybridizable to the 3′ end of exon 1A and a second continuous region of at least 5 to 15 nucleotides that is complementary and hybridizable to the 5′ end of exon 8. A large number of different polynucleotide sequences from the region of the exon 1A to exon 8 splice junction may be selected which will, under appropriate hybridization conditions, have the capacity to detectably hybridize to PDE4Bsv1 polynucleotide and yet will hybridize to a much less extent or not at all to PDE4B isoform polynucleotides wherein exon 1A is not spliced to exon 8.

Preferably, non-complementary nucleic acid that is present has a particular purpose such as being a reporter sequence or being a capture sequence. However, additional nucleic acid need not have a particular purpose as long as the additional nucleic acid does not prevent the PDE4Bsv1 nucleic acid from distinguishing between target polynucleotides, e.g., PDE4Bsv1 polynucleotides, and non-target polynucleotides, including, but not limited to PDE4B polynucleotides not comprising the exon 1A to exon 8 splice junction found in PDE4Bsv1.

Hybridization occurs through complementary nucleotide bases. Hybridization conditions determine whether two molecules, or regions, have sufficiently strong interactions with each other to form a stable hybrid.

The degree of interaction between two molecules that hybridize together is reflected by the melting temperature (Tₘ) of the produced hybrid. The higher the Tₘ the stronger the interactions and the more stable the hybrid. Tₘ is effected by different factors well known in the art such as the degree of complementarity, the type of complementary bases present (e.g., A-T hybridization versus G-C hybridization), the presence of modified nucleic acid, and solution components (e.g., Sambrook, et al., in Molecular Cloning, A Laboratory Manual, 2^{nd} Edition, Cold Spring Harbor Laboratory Press, 1989).

Stable hybrids are formed when the Tₘ of a hybrid is greater than the temperature employed under a particular set of hybridization assay conditions. The degree of specificity of a probe can be varied by adjusting the hybridization stringency conditions. Detecting probe hybridization is facilitated through the use of a detectable label. Examples of detectable labels include luminescent, enzymatic, and radioactive labels.

Examples of stringency conditions are provided in Sambrook, et al., in Molecular Cloning, A Laboratory Manual, 2^{nd} Edition, Cold Spring Harbor Laboratory Press, 1989. An example of high stringency conditions is as follows: Prehybridization of filters containing DNA is carried out for 2 hours to overnight at 65° C. in buffer composed of 6×SSC, 5×Denhardt's solution, and 100 μg/ml denatured salmon sperm DNA. Filters are hybridized for 12 to 48 hours at 65° C. in prehybridization mixture containing 100 μg/ml denatured salmon sperm DNA and 5−20×10⁶ cpm of ³²P-labeled probe. Filter washing is done at 37° C. for 1 hour in a solution containing 2×SSC, 0.1% SDS. This is followed by a wash in 0.1×SSC, 0.1% SDS at 50° C. for 45 minutes before autoradiography. Other procedures using conditions of high stringency would include, for example, either a hybridization step carried out in 5×SSC, 5×Denhardt's solution, 50% formamide at 42° C. for 12 to 48 hours or a washing step carried out in 0.2×SSPE, 0.2% SDS at 65° C. for 30 to 60 minutes.

Recombinant Expression

PDE4Bsv1 polynucleotides, such as those comprising SEQ ID NO:3, can be used to make PDE4Bsv1 polypeptides. In particular, PDE4Bsv1 polypeptides can be expressed from recombinant nucleic acids in a suitable host or in vitro using a translation system. Recombinantly expressed PDE4Bsv1 polypeptides can be used, for example, in assays to screen for compounds that bind PDE4Bsv1. Alternatively, PDE4Bsv1 polypeptides can also be used to screen for compounds that bind to one or more PDE4B or PDE isoforms, but do not bind to PDE4Bsv1.

In some embodiments, expression is achieved in a host cell using an expression vector. An expression vector contains recombinant nucleic acid encoding a polypeptide along with regulatory elements for proper transcription and processing. The regulatory elements that may be present include those naturally associated with the recombinant nucleic acid and exogenous regulatory elements not naturally associated with the recombinant nucleic acid. Exogenous regulatory elements such as an exogenous promoter can be useful for expressing recombinant nucleic acid in a particular host.

Generally, the regulatory elements that are present in an expression vector include a transcriptional promoter, a ribosome binding site, a terminator, and an optionally present operator. Another preferred element is a polyadenylation signal providing for processing in eukaryotic cells. Preferably, an expression vector also contains an origin of replication for autonomous replication in a host cell, a selectable marker, a limited number of useful restriction enzyme sites, and a potential for high copy number. Examples of expression vectors are cloning vectors, modified cloning vectors, and specifically designed plasmids and viruses.

Expression vectors providing suitable levels of polypeptide expression in different hosts are well known in the art. Mammalian expression vectors well known in the art include, but are not restricted to, pcDNA3 (Invitrogen, Carlsbad Calif.), pSecTag2 (Invitrogen), pMC1 neo (Stratagene, La Jolla Calif.), pXT1 (Stratagene), pSG5 (Stratagene), pCMVLacI (Stratagene), pCI-neo (Promega), EBO-pSV2-neo (ATCC 37593), pBPV-1(8-2) (ATCC 37110), pdBPV-MMTneo(342-12) (ATCC 37224), pRSVgpt (ATCC 37199), pRSVneo (ATCC 37198), pSV2-dhfr (ATCC 37146) and pUCTag (ATCC 37460). Bacterial expression vectors well known in the art include pET11a (Novagen), pBluescript SK (Stratagene, La Jolla), pQE-9 (Qiagen Inc., Valencia), lambda gt11 (Invitrogen), pcDNAII (Invitrogen), and pKK223-3 (Pharmacia). Fungal cell expression vectors well known in the art include pRS416 (ATCC 87521), pPICZ (Invitrogen), pYES2 (Invitrogen), and Pichia expression vector (Invitrogen). Insect cell expression vectors well known in the art include Blue Bac III (Invitrogen), pBacPAK8 (CLONTECH, Inc., Palo Alto) and PfastBacHT (Invitrogen, Carlsbad, Calif.).

Recombinant host cells may be prokaryotic or eukaryotic. Examples of recombinant host cells include the following: bacteria such as E. coli; fungal cells such as yeast; mammalian cells such as human, bovine, porcine, monkey and rodent; and insect cells such as Drosophila and silkworm derived cell lines. Commercially available mammalian cell lines include L cells L-M(TK⁻) (ATCC CCL 1.3), L cells L-M (ATCC CCL 1.2), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C1271 (ATCC CRL 1616), BS-C-1 (ATCC CCL 26) MRC-5 (ATCC CCL 171), and HEK 293 cells (ATCC CRL-1573).

To enhance expression in a particular host it may be useful to modify the sequence provided in SEQ ID NO:3 to take into account codon usage of the host. Codon usages of different organisms are well known in the art (see, Ausubel, Current Protocols in Molecular Biology, John Wiley, 1987-1998, Supplement 33 Appendix 1C).

Expression vectors may be introduced into host cells using standard techniques. Examples of such techniques include transformation, transfection, lipofection, protoplast fusion, and electroporation.

Nucleic acids encoding for a polypeptide can be expressed in a cell without the use of an expression vector employing, for example, synthetic mRNA or native mRNA. Additionally, mRNA can be translated in various cell-free systems such as wheat germ extracts and reticulocyte extracts, as well as in cell based systems, such as frog oocytes. Introduction of mRNA into cell based systems can be achieved, for example, by microinjection or electroporation.

PDE4Bsv1 Polypeptides

PDE4Bsv1 polypeptides contain an amino acid sequence comprising, consisting or consisting essentially of SEQ ID NO:4. PDE4Bsv1 polypeptides have a variety of uses, such as providing a marker for the presence of PDE4Bsv1; use as an immunogen to produce antibodies binding to PDE4Bsv1; use as a target to identify compounds binding selectively to PDE4Bsv1; or use in an assay to identify compounds that bind to one or more PDE4B or PDE isoforms but do not bind to or interact with PDE4Bsv1.

In chimeric polypeptides containing one or more regions from PDE4Bsv1 and one or more regions not from PDE4Bsv1, the region(s) not from PDE4Bsv1 can be used, for example, to achieve a particular purpose or to produce a polypeptide that can substitute for PDE4Bsv1, or fragments thereof. Particular purposes that can be achieved using chimeric PDE4Bsv1 polypeptides include providing a marker for PDE4Bsv1 activity, enhancing an immune response, and altering the activity and regulation of PDE4B.

Polypeptides can be produced using standard techniques including those involving chemical synthesis and those involving biochemical synthesis. Techniques for chemical synthesis of polypeptides are well known in the art (see e.g., Vincent, in Peptide and Protein Drug Delivery, New York, N.Y., Dekker, 1990).

Biochemical synthesis techniques for polypeptides are also known in the art. Such techniques employ a nucleic acid template for polypeptide synthesis. The genetic code providing the sequences of nucleic acid triplets coding for particular amino acids is well known in the art (see, e.g., Lewin GENES IV, p. 119, Oxford University Press, 1990). Examples of techniques for introducing nucleic acid into a cell and expressing the nucleic acid to produce protein are provided in references such as Ausubel, Current Protocols in Molecular Biology, John Wiley, 1987-1998, and Sambrook, et al., in Molecular Cloning, A Laboratory Manual, 2^{nd} Edition, Cold Spring Harbor Laboratory Press, 1989.

Functional PDE4Bsv1

Functional PDE4Bsv1 is a different protein isoform of PDE4B. The identification of the amino acid and nucleic acid sequences of PDE4Bsv1 provides tools for obtaining functional proteins related to PDE4Bsv1 from other sources, for producing PDE4Bsv1 chimeric proteins, and for producing functional derivatives of SEQ ID NO: 4.

PDE4Bsv1 polypeptides can be readily identified and obtained based on their sequence similarity to PDE4Bsv1 (SEQ ID NO:4). In particular, PDE4Bsv1 lacks the amino acids encoded by exons 1-7 of the PDE4B gene and retains an alternative exon 1A deriving from the sequence of intron 7. The precise 5′ end of exon 1A has not been determined; however, 102 base pairs of sequence at the 3′ end of exon 1A are presented in SEQ ID NO:2. The PDE4Bsv1 polypeptide also initiates at an alternative start codon in exon 1A, located 48 nucleotides from the 3′ end of exon 1A and SEQ ID NO:2. The replacement of exons 1-7 with exon 1A and the use of alternative start codon in exon 1A do not alter the protein translation reading frame as compared to the PDE4B reference sequence (NM_{—}002600). Thus, the PDE4Bsv1 polypeptide is lacking the amino acids encoded by the nucleotides corresponding to exon 1-7 of the PDE4B reference transcript (NM_{—}002600) and possesses a unique N-terminal 16 amino acid region encoded by the nucleotides corresponding to exon 1A. PDE4Bsv1 posses an intact catalytic domain. Based upon the identification of other active PDE4 splice variants which have truncated UCR regions, PDE4Bsv1 is expected to have activity (Bolger et al., 1994, Gene 149:237-244; Sullivan et al., 1998, Biochem. J. 333:693-703; Huston et al. 1997, Biochem. J. 328:549-558).

Both the amino acid and nucleic acid sequences of PDE4Bsv1 can be used to help identify and obtain PDE4Bsv1 polypeptides. For example, SEQ ID NO:3 can be used to produce degenerative nucleic acid probes or primers for identifying and cloning nucleic acid polynucleotides encoding for an PDE4Bsv1 polypeptide. In addition, polynucleotides comprising, consisting, or consisting essentially of SEQ ID NO:3 or fragments thereof, can be used under conditions of moderate stringency to identify and clone nucleic acids encoding PDE4Bsv1 polypeptides from a variety of different organisms.

The use of degenerative probes and moderate stringency conditions for cloning is well known in the art. Examples of such techniques are described by Ausubel, Current Protocols in Molecular Biology, John Wiley, 1987-1998, and Sambrook, et al., in Molecular Cloning, A Laboratory Manual, 2^{nd} Edition, Cold Spring Harbor Laboratory Press, 1989.

Starting with PDE4Bsv1 obtained from a particular source, derivatives can be produced. Such derivatives include polypeptides with amino acid substitutions, additions and deletions. Changes to PDE4Bsv1 to produce a derivative having essentially the same properties should be made in a manner not altering the tertiary structure of PDE4Bsv1.

Differences in naturally occurring amino acids are due to different R groups. An R group affects different properties of the amino acid such as physical size, charge, and hydrophobicity. Amino acids are can be divided into different groups as follows: neutral and hydrophobic (alanine, valine, leucine, isoleucine, proline, tryptophan, phenylalanine, and methionine); neutral and polar (glycine, serine, threonine, tryosine, cysteine, asparagine, and glutamine); basic (lysine, arginine, and histidine); and acidic (aspartic acid and glutamic acid).

Generally, in substituting different amino acids it is preferable to exchange amino acids having similar properties. Substituting different amino acids within a particular group, such as substituting valine for leucine, arginine for lysine, and asparagine for glutamine are good candidates for not causing a change in polypeptide functioning.

Changes outside of different amino acid groups can also be made. Preferably, such changes are made taking into account the position of the amino acid to be substituted in the polypeptide. For example, arginine can substitute more freely for nonpolar amino acids in the interior of a polypeptide then glutamate because of its long aliphatic side chain (See, Ausubel, Current Protocols in Molecular Biology, John Wiley, 1987-1998, Supplement 33 Appendix 1C).

PDE4Bsv1 Antibodies

Antibodies recognizing PDE4Bsv1 can be produced using a polypeptide containing SEQ ID NO: 4, or a fragment thereof as an immunogen. Preferably, an PDE4Bsv1 polypeptide used as an immunogen consists of a polypeptide of SEQ ID NO:4 or a SEQ ID NO:4 fragment having at least 10 contiguous amino acids in length corresponding to the polynucleotide region representing the junction resulting from the splicing of exon 1A to exon 8 of the PDE4B gene.

In some embodiments where, for example, PDE4Bsv1 polypeptides are used to develop antibodies that bind specifically to PDE4Bsv1 and not to other isoforms of PDE4B, the PDE4Bsv1polypeptides comprise at least 10 amino acids of the PDE4Bsv1 polypeptide sequence corresponding to a junction polynucleotide region created by the alternative splicing of exon 1A to exon 8 of the primary transcript of the PDE4B gene (see FIG. 1). For example, the amino acid sequence: amino terminus-WGYIKFKRML-carboxy terminus (SEQ ID NO: 6) represents one embodiment of such an inventive PDE4Bsv1 polypeptide wherein a first 5 amino acid region is encoded by a nucleotide sequence at the 3′ end of exon 1A of the PDE4B gene and a second 5 amino acid region is encoded by the nucleotide sequence directly after the novel splice junction. Preferably, at least 10 amino acids of the PDE4Bsv1polypeptide comprise a first continuous region of 2 to 8 amino acids that is encoded by nucleotides at the 3′ end of exon 1A and a second continuous region of 2 to 8 amino acids that is encoded by nucleotides at the 5′ end of exon 8.

In other embodiments, PDE4Bsv 1-specific antibodies are made using a PDE4Bsv1polypeptide that comprises at least 20, 30, 40 or 50 amino acids of the PDE4Bsv1 sequence that corresponds to ajunction polynucleotide region created by the alternative splicing of exon 1A to exon 8 of the primary transcript of the PDE4B gene. In each case the PDE4Bsv1 polypeptides are selected to comprise a first continuous region of at least 5 to 15 amino acids that is encoded by nucleotides at the 3′ end of exon 1A and a second continuous region of 5 to 15 amino acids that is encoded by nucleotides directly after the novel splice junction.

Antibodies to PDE4Bsv1 have different uses, such as to identify the presence of PDE4Bsv1, and to isolate PDE4Bsv1 polypeptides. Identifying the presence of PDE4Bsv1 can be used, for example, to identify cells producing PDE4Bsv1. Such identification provides an additional source of PDE4Bsv1 and can be used to distinguish cells known to produce PDE4Bsv1 from cells that do not produce PDE4Bsv1. For example, antibodies to PDE4Bsv1 can distinguish human cells expressing PDE4Bsv1 from human cells not expressing PDE4Bsv1 or non-human cells (including bacteria) that do not express PDE4Bsv1. Such PDE4Bsv1 antibodies can also be used to determine the effectiveness of PDE4Bsv1 ligands, using techniques well known in the art, to detect and quantify changes in the protein levels of PDE4Bsv1 in cellular extracts, and in situ immunostaining of cells and tissues.

Techniques for producing and using antibodies are well known in the art. Examples of such techniques are described in Ausubel, Current Protocols in Molecular Biology, John Wiley, 1987-1998; Harlow, et al., Antibodies, A Laboratory Manual, Cold Spring Harbor Laboratory, 1988; and Kohler, et al., 1975 Nature 256:495-7.

PDE4Bsv1 Binding Assay

A number of compounds known to modulate PDE4B activity have been disclosed. Rolipram, roflumilast, and cilomilast act as inhibitors of PDE4B function (Schwabe et al., 1976, Mol. Pharmacol. 12:900-910; Hatzelmann and Schudt, 2001, J. Pharmacol. Exp. Ther. 297:267-279; Barnette et al., 1998, J. Pharmacol. Exp. Ther. 284: 420-426). Additional PDE4 inhibitor compounds have also been disclosed (US2006/0041006; US2006/0100218; U.S. Pat. No. 6,841,564; U.S. Pat. No. 6,740,666; U.S. Pat. No. 6,200,993). Methods for monitoring the ligand binding activity of PDE4B, including analyzing the effect of compounds on the ligand binding activity of PDE4B, have been described previously (US2006/0100218). Methods for screening compounds for their effects on PDE4B activity have also been described (WO02/086152). A person skilled in the art should be able to use these methods to screen PDE4Bsv1 polypeptide for compounds that bind to, and in some cases functionally alter, PDE4B isoform proteins.

PDE4Bsv1 or fragments thereof, can be used in binding studies to identify compounds binding to or interacting with PDE4Bsv1, or fragments thereof. In one embodiment, PDE4Bsv1, or a fragment thereof, can be used in binding studies with a PDE isoform protein, or a fragment thereof, to identify compounds that: bind to or interact with PDE4Bsv1 and other PDE isoforms; bind to or interact with one or more other PDE isoforms and not with PDE4Bsv1; bind to or interact with PDE4Bsv1 and not with one or more other PDE isoforms. Such binding studies can be performed using different formats including competitive and non-competitive formats. Further competition studies can be carried out using additional compounds determined to bind to PDE4Bsv1, other PDE4, or other PDE isoforms.

The particular PDE4Bsv1 sequence involved in ligand binding can be identified using labeled compounds that bind to the protein and different protein fragments. Different strategies can be employed to select fragments to be tested to narrow down the binding region. Examples of such strategies include testing consecutive fragments about 15 amino acids in length starting at the N-terminus, and testing longer length fragments. If longer length fragments are tested, a fragment binding to a compound can be subdivided to further locate the binding region. Fragments used for binding studies can be generated using recombinant nucleic acid techniques.

In some embodiments, binding studies are performed using PDE4Bsv1 expressed from a recombinant nucleic acid. Alternatively, recombinantly expressed PDE4Bsv1 consists of the SEQ ID NO:4 amino acid sequence.

Binding assays can be performed using individual compounds or preparations containing different numbers of compounds. A preparation containing different numbers of compounds having the ability to bind to PDE4Bsv1 can be divided into smaller groups of compounds that can be tested to identify the compound(s) binding to PDE4Bsv1.

Binding assays can be performed using recombinantly produced PDE4Bsv1 present in different environments. Such environments include, for example, cell extracts and purified cell extracts containing a PDE4Bsv1 recombinant nucleic acid; and also include, for example, the use of a purified PDE4Bsv1 polypeptide produced by recombinant means which is introduced into different environments.

In one embodiment of the invention, a binding method is provided for screening for a compound able to bind selectively to PDE4Bsv1. The method comprises the steps: providing a PDE4Bsv1 polypeptide comprising SEQ ID NO:4; providing an PDE isoform polypeptide that is not PDE4Bsv1; contacting the PDE4Bsv1 polypeptide and the PDE isoform polypeptide that is not PDE4Bsv1 with a test preparation comprising one or more test compounds; and then determining the binding of the test preparation to the PDE4Bsv1 polypeptide and to the PDE isoform polypeptide that is not PDE4Bsv1, wherein a test preparation that binds to the PDE4Bsv1 polypeptide, but does not bind to the PDE isoform polypeptide that is not PDE4Bsv1, contains one or more compounds that selectively bind to PDE4Bsv1.

In another embodiment of the invention, a binding method is provided for screening for a compound able to bind selectively to a PDE isoform polypeptide that is not PDE4Bsv1. The method comprises the steps: providing a PDE4Bsv1 polypeptide comprising SEQ ID NO:4; providing an PDE isoform polypeptide that is not PDE4Bsv1; contacting the PDE4Bsv1 polypeptide and the PDE isoform polypeptide that is not PDE4Bsv1 with a test preparation comprising one or more test compounds; and then determining the binding of the test preparation to the PDE4Bsv1 polypeptide and the PDE isoform polypeptide that is not PDE4Bsv1, wherein a test preparation that binds the PDE isoform polypeptide that is not PDE4Bsv1, but does not bind PDE4Bsv1, contains a compound that selectively binds the PDE isoform polypeptide that is not PDE4Bsv1.

The above-described selective binding assays can also be performed with a polypeptide fragment of PDE4Bsv1, wherein the polypeptide fragment comprises at least 10 consecutive amino acids that are coded by a nucleotide sequence that bridges the junction created by the splicing of the 3′ end of exon 1A to the 5′ end of exon 8 in the case of PDE4Bsv1. Similarly, the selective binding assays may also be performed using a polypeptide fragment of a PDE isoform polypeptide that is not PDE4Bsv1, wherein the polypeptide fragment comprises at least 10 consecutive amino acids that are coded by: a) a nucleotide sequence that is contained within exons 1-7 of the PDE4B gene or b) a nucleotide sequence that bridges the junction created by the splicing of the 3′ end of exon 1 to the 5′ end of exon 2 of the PDE4B gene; or the junction created by the splicing of the 3′ end of exon 2 to the 5′ end of exon 3; or the junction created by the splicing of the 3′ end of exon 3 to the 5′ end of exon 4; or the junction created by the splicing of the 3′ end of exon 4 to the 5′ end of exon 5; or the junction created by the splicing of the 3′ end of exon 5 to the 5′ end of exon 6; or the junction created by the splicing of the 3′ end of exon 6 to the 5′ end of exon 7; or the junction created by the splicing of the 3′ end of exon 7 to the 5′ end of exon 8.

PDE4B Functional Assays

PDE4B encodes phosphodiesterase 4B, an important component of cAMP regulation, that is implicated in psychiatric, memory, and inflammatory disorders such as schizophrenia, depression, asthma, and COPD. Splice variants of PDE may exhibit different catalytic activity and different binding affinities for compounds, peptides, or other small molecules. The identification of PDE4Bsv1 as a splice variant of PDE4B provides a means of screening for compounds that bind to PDE4Bsv1 protein thereby altering the activity or regulation of PDE4Bsv1. Assays involving a functional PDE4Bsv1 polypeptide can be employed for different purposes, such as selecting for compounds active at PDE4Bsv1; evaluating the ability of a compound to affect the activity of each respective splice variant; and mapping the activity of different PDE4Bsv1 regions. PDE4Bsv1 activity can be measured using different techniques such as: detecting a change in the intracellular conformation of PDE4Bsv1; detecting a change in the intracellular location of PDE4Bsv1; or measuring the phosphodiesterase activity of PDE4Bsv1.

Recombinantly expressed PDE4Bsv1 can be used to facilitate the determination of whether a compound's activity in a cell is dependent upon the presence of PDE4Bsv1. For example, PDE4Bsv1 can be expressed by an expression vector in a cell line and used in a co-culture growth assay, such as described in U.S. Pat. No. 6,518,035, to identify compounds that alter the growth of the cell expressing PDE4Bsv1 from the expression vector as compared to the same cell line but lacking the PDE4Bsv1expression vector. Alternatively, determination of whether a compound's activity on a cell is dependent upon the presence of PDE4Bsv1 can also be done using gene expression profile analysis methods as described, for example, in U.S. Pat. No. 6,324,479.

Methods to determine PDE4B activity are known in the art. A radiochemical method that measures hydrolysis of [³H]cAMP to [³H]AMP has been described (Claveau et al. 2004, J. Pharmacol. Exp. Ther. 310:752-760; Laliberte et al., 2000, Biochemistry 39:6449-6458; Bolger et al., 1993, Mol. Cell. Biol. 13:6558-6571; Marchmont et al., 1980, Biochem. J. 187:381-392; Shepard et al., 2004, Br. J. Pharmacology 142:339-351). Methods for expressing PDE4 enzymes in E. coli, insect cells, and CHO-K1 cells and monitoring the activity of PDE4, including analyzing the effect of compounds on PDE4 activity, have been described previously (U.S. Patent Application 2006/100218; U.S. Pat. No. 5,922,557). A variety of other assays has been used to investigate the properties of PDE4 and PDE4B, and therefore, would also be applicable to the measurement of PDE4Bsv1.

In one embodiment of the invention, a screening method is provided for screening a compound that modulates the activity of PDE4Bsv1. The method comprises: expressing a recombinant nucleic acid encoding PDE4Bsv1 comprising SEQ ID NO:4 in a cell; contacting said cell or a cell extract thereof with a test preparation comprising one or more test compounds; and then measuring the effect of said test preparation on enzyme activity. PDE4Bsv1 functional assays can be performed using cells expressing PDE4Bsv1 at a high level. These proteins will be contacted with individual compounds or test preparations containing different compounds. A test preparation containing different compounds where one or more compounds affect PDE4Bsv1 in cells over-producing PDE4Bsv1 as compared to control cells containing an expression vector lacking PDE4Bsv1 coding sequences, can be divided into smaller groups of compounds to identify the compound(s) affecting PDE4Bsv1 activity.

PDE4Bsv1 functional assays can be performed using recombinantly produced PDE4Bsv1 present in different environments. Such environments include, for example, cell extracts and purified cell extracts containing the PDE4Bsv1 expressed from recombinant nucleic acid; and the use of purified PDE4Bsv1 produced by recombinant means that is introduced into a different environment suitable for measuring PDE4B activity.

Modulating PDE4Bsv1 Expression

PDE4Bsv1 expression can be modulated as a means for increasing or decreasing PDE4Bsv1 activity. Such modulation includes inhibiting the activity of nucleic acids encoding the PDE4B isoform target to reduce PDE4B isoform protein or polypeptide expression, or supplying PDE4B nucleic acids to increase the level of expression of the PDE4B target polypeptide thereby increasing PDE4B activity.

Inhibition of PDE4Bsv1 Activity

PDE4Bsv1 nucleic acid activity can be inhibited using nucleic acids recognizing PDE4Bsv1 nucleic acid and affecting the ability of such nucleic acid to be transcribed or translated. Inhibition of PDE4Bsv1 nucleic acid activity can be used, for example, in target validation studies.

A preferred target for inhibiting PDE4Bsv1 is mRNA stability and translation. The ability of PDE4Bsv1 mRNA to be translated into a protein can be effected by compounds such as anti-sense nucleic acid, RNA interference (RNAi) and enzymatic nucleic acid.

Anti-sense nucleic acid can hybridize to a region of a target mRNA. Depending on the structure of the anti-sense nucleic acid, anti-sense activity can be brought about by different mechanisms such as blocking the initiation of translation, preventing processing of mRNA, hybrid arrest, and degradation of mRNA by RNAse H activity.

RNA inhibition (RNAi) using shRNA or siRNA molecules can also be used to prevent protein expression of a target transcript. This method is based on the interfering properties of double-stranded RNA derived from the coding region of a gene that disrupts the synthesis of protein from transcribed RNA.

Enzymatic nucleic acids can recognize and cleave other nucleic acid molecules. Preferred enzymatic nucleic acids are ribozymes.

General structures for anti-sense nucleic acids, RNAi and ribozymes, and methods of delivering such molecules, are well known in the art. Modified and unmodified nucleic acids can be used as anti-sense molecules, RNAi and ribozymes. Different types of modifications can affect certain RNA activities such as the ability to be cleaved by RNAse H, and can affect nucleic acid stability. Examples of references describing different anti-sense molecules, and ribozymes, and the use of such molecules, are provided in U.S. Pat. Nos. 5,849,902; 5,859,221; 5,852,188; and 5,616,459. Examples of organisms in which RNAi has been used to inhibit expression of a target gene include: C. elegans (Tabara, et al., 1999, Cell 99:123-32; Fire, et al., 1998, Nature 391:806-11), plants (Hamilton and Baulcombe, 1999, Science 286:950-52), Drosophila (Hammond, et al., 2001, Science 293:1146-50; Misquitta and Patterson, 1999, Proc. Nat. Acad. Sci. 96, 1451-56; Kennerdell and Carthew, 1998, Cell 95:1017-26), and mammalian cells (Bernstein, et al., 2001, Nature 409, 363-6; Elbashir, et al., 2001, Nature 411:494-8).

Increasing PDE4Bsv1 Expression

Nucleic acids encoding for PDE4Bsv1 can be used, for example, to cause an increase in PDE4B activity or to create a test system (e.g., a transgenic animal) for screening for compounds affecting PDE4Bsv1 expression, respectively. Nucleic acids can be introduced and expressed in cells present in different environments.

Guidelines for pharmaceutical administration in general are provided in, for example, Remington's Pharmaceutical Sciences, 18^{th} Edition, supra, and Modern Pharmaceutics, 2^{nd} Edition, supra Nucleic acid can be introduced into cells present in different environments using in vitro, in vivo, or ex vivo techniques. Examples of techniques useful in gene therapy are illustrated in Gene Therapy & Molecular Biology From Basic Mechanisms to Clinical Applications, Ed. Boulikas, Gene Therapy Press, 1998.

### EXAMPLES

Examples are provided below to further illustrate different features and advantages of the present invention. The examples also illustrate useful methodology for practicing the invention. These examples do not limit the claimed invention.

### Example 1

### Identification of PDE4Bsv1 Using Genomically Aligned ESTs and RT-PCR

Using computational and experimental methods, an alternatively spliced isoform of PDE4B was identified. Alternative splicing analysis of the PDE4B gene was performed by aligning expressed sequence tags (EST) to the genomic sequence, using a cross-species approach. Methods for gene structure prediction using genomically aligned ESTs are known in the art and have been described (Mironov et al., 1999, Genome Res. 9:1288-1293; Kan et al., 2001, Genome Res. 11:889-900; Kan et al., 2002, Genome Res. 12:1837-1845; Modrek et al., 2001, Nucleic Acids Res. 29:2850-2859). Detection of novel splice forms in human and mouse using a cross-species approach was conducted as previously described (Kan et al., 2004, Pac. Symp. Biocomputing 9:42-53). The mRNA transcript sequence for PDE4B (NM_{—}002600) and human PDE4B EST sequences were aligned to the human PDE4B genomic sequence using the sim4 alignment program (Florea et al., 1998; Genome Res. 8:967-974), which allows for introns in the genomic sequence and a small number of sequencing errors. The Transcript Assembly Program (TAP, Kan et al., 2001, Genome Res. 11:889-900) was used to predict the gene structure from the genomic EST alignment and compare the predicted gene structures with the known gene structures. Consensus splice patterns were similarly constructed for mouse PDE4B. In the second phase, cross-species alignments were generated by aligning the mouse consensus sequence to the human genome. Cross-species alignments are then used to identify alternative splice patterns using TAP. Mouse EST (BQ769324) was identified as containing a splicing pattern different from the PDE4B mRNA transcript NM_{—}002600. Mouse EST (BQ769324) contains an exon 1 not found in NM_{—}002600 or any other known PDE4B mRNA sequences. This novel PDE4B splice isoform was predicted to have used an alternative exon 1 (exon 1A), located within intron 7 of the PDE4B gene.

To test this computational prediction of a novel PDE4B splice isoform in humans, the structure of PDE4B mRNA in the region corresponding to exon 1A to exon 10, which encompasses the unique N-terminal domain of PDE4Bsv1, was determined for a panel of 44 human tissues and cell line samples using an RT-PCR based assay. PolyA purified mRNA isolated 44 different human tissue and cell line samples was obtained from BD Biosciences Clontech (Palo Alto, Calif.). RT-PCR primers were selected that were complementary to sequences in exon 1A and exon 10 of the mouse EST (BQ769324), and reference exon coding sequences in PDE4B (NM_{—}002600), respectively. Based upon the computational prediction of a novel PDE4B splice isoform, the PDE4B exon 1A and exon 10 primer set (hereafter PDE4B_{1A-10} primer set) was expected to amplify a 442 base pair amplicon representing the PDE4B mRNA region of the predicted alternatively spliced isoform. The PDE4B exon 1A forward primer has the sequence: 5′ ACTGTGAATTCTTTCAAAGGGATTTGTG 3′ (SEQ ID NO 7); and the PDE4B exon 10 reverse primer has the sequence: 5′ GGTCTATTGTGAGAATATCCAGCCACAT 3′ (SEQ ID NO 8).

Twenty-five ng of polyA mRNA from each tissue was subjected to a one-step reverse transcription-PCR amplification protocol using the Qiagen, Inc. (Valencia, Calif.), One-Step RT-PCR kit, using the following cycling conditions:

50° C. for 30 minutes;

95° C. for 15 minutes;

35 cycles of:
94° C. for 30 seconds;
63.5° C. for 40 seconds;
72° C. for 50 seconds; then
72° C. for 10 minutes.

RT-PCR amplification products (amplicons) were size fractionated on a 2% agarose gel. Selected amplicon fragments were manually extracted from the gel and purified with a Qiagen Gel Extraction Kit. Purified amplicon fragments were cloned into an Invitrogen pCR2.1 vector using the reagents and instructions provided with the TOPO TA cloning kit (Invitrogen, Carlsbad, Calif.). Clones were then sequenced from each end (using the same primers used for RT-PCR) by Qiagen Genomics, Inc. (Bothell, Wash.).

The RT-PCR amplicons obtained from human retina, pituitary, spinal cord, brain tissues, fetal brain, fetal kidney, and lung carcinoma polyA mRNA samples using the PDE4B_{1A-10} primer set exhibited the expected amplicon size of 442 base pairs for the alternatively spliced PDE4B mRNA (data not shown).

Sequence analysis of the about 442 base pair amplicon revealed that this amplicon form results from the deletion of exons 1-7 of the PDE4B heteronuclear RNA (hnRNA) and the retention of sequence from intron 7, forming a novel 5′ exon, referred to as exon 1A. This splice variant form was designated PDE4Bsv1 (SEQ ID NO: 3). Thus, the RT-PCR results suggested that PDE4B mRNA in some tissue samples is composed of a population of molecules wherein in at least one of the PDE4B mRNA splice junctions is altered.

### Example 2

### Cloning of PDE4Bsv1

Computational prediction, RT-PCR, and sequencing data indicate that in addition to the normal PDE4B reference mRNA sequence, NM_{—}002600, encoding PDE4B protein, NP_{—}002591, a novel splice variant form of PDE4B mRNA also exists in retina, pituitary, spinal cord, fetal kidney, lung carcinoma, and brain tissues.

Method 1:

Clones having a nucleotide sequence comprising the splice variant identified in Example 1 (hereafter referred to as PDE4Bsv1) are isolated using a 5′ “forward” PDE4Bsv1 primer and a 3′ “reverse” PDE4Bsv1 primer, to amplify and clone the entire PDE4Bsv1 mRNA coding sequences. The 5′ “forward” primer is designed for isolation of full length clones corresponding to the PDE4Bsv1 splice variant and has the nucleotide sequence of 5′ AGATGGCTGTGTTTCCTAGTCTGGCAACTCC 3′ (SEQ ID NO: 9). The 3′ “reverse” primer is designed for isolation of full length clones corresponding to the PDE4Bsv1 splice variant and has the nucleotide sequence of 5′TTATGTATCCACGGGGGACTTGTCTTCTGTTGC 3′ (SEQ ID NO: 10).

RT-PCR

The PDE4Bsv1 cDNA sequence is cloned using a combination of reverse transcription (RT) and polymerase chain reaction (PCR), using the Titan One Tube RT-PCR Kit (Roche Applied Science, Indianapolis, Ind.). More specifically, about 25 ng of brain tissue polyA mRNA (BD Biosciences Clontech, Palo Alto, Calif.) is reverse transcribed using AMV Reverse Transcriptase and amplified using the Expand High Fidelity enzyme mixture in a one step reaction system according to the Titan One Tube RT-PCR Kit manufacturer's instructions. Reactions components are set up as two separate Master Mixes. Master Mix 1 contains the following components final concentrations in a 25 μl total reaction volume: 0.2 mM dNTPs (each), 5 mM DTT solution, 5 U RNase Inhibitor, 0.4 μM PDE4Bsv1 “forward” primer (SEQ ID NO: 9), 0.4 μM PDE4Bsv1 “reverse” primer (SEQ ID NO: 10), 25 ng brain tissue RNA, and sterile water to 25 μl final volume. Master Mix 2 contains the following components in a 25 μl total reaction volume: 14 μl sterile water, 10 μl 5×RT-PCR buffer, and 1 μl enzyme mix. 25 μl of each Master Mix 1 and 2 are combined and placed on ice. For the RT step, the sample is placed in a thermocycler for 30 minutes at 48° C. The RT step is followed by a thermocycling step, which is done in a Gene Amp PCR System 9700 (Applied Biosystems, Foster City, Calif.). After an initial 94° C. denaturation of 2 minutes, 10 cycles of amplification are performed using a 30 second denaturation at 94° C. followed by a 30 second annealing at 63.5° C., and a 5 minute synthesis at 68° C. The 10 cycles of PCR are followed by an additional 25 cycles of a 30 second denaturation at 94° C., followed by a 30 second annealing at 63.5° C., and a 5 minute synthesis at 68° C.+cycle elongation of 5 seconds for each successive cycle (i.e., cycle 11 has an additional 5 seconds, cycle 12 has an additional 10 seconds). The additional 15 cycles are followed by a 7 minute extension at 68° C. The 50 μl reaction is then chilled to 4° C. 10 μl of the resulting reaction product is run on a 1% agarose (Invitrogen, Ultra pure) gel stained with 0.3 μg/ml ethidium bromide (Fisher Biotech, Fair Lawn, N.J.). Nucleic acid bands in the gel are visualized and photographed on a UV light box to determine if the PCR had yielded products of the expected size, in the case of the predicted PDE4Bsv1 mRNA, a product of about 1953 base pairs. The remainder of the 50 μl PCR reactions from adipose tissue is purified using the QIAquik Gel extraction Kit (Qiagen, Valencia, Calif.) following the QIAquik PCR Purification Protocol provided with the kit. About 50 μl of product obtained from the purification protocol is concentrated to about 6 μl by drying in a Speed Vac Plus (SC110A, from Savant, Holbrook, N.Y.) attached to a Universal Vacuum System 400 (also from Savant) for about 30 minutes on medium heat.

Cloning of RT-PCR Products

About 4 μl of the 6 μl of purified PDE4Bsv1 RT-PCR product from brain tissue are used in a cloning reaction using the reagents and instructions provided with the pCR8/GW/TOPO TA cloning kit (Invitrogen, Carlsbad, Calif.). About 2 μl of the cloning reaction is used following the manufacturer's instructions to transform TOP10 chemically competent E. coli provided with the cloning kit. After the 1 hour recovery of the cells in SOC medium (provided with the TOPO TA cloning kit), 200 μl of the mixture is plated on LB medium plates (Sambrook, et al., in Molecular Cloning, A Laboratory Manual, 2^{nd} Edition, Cold Spring Harbor Laboratory Press, 1989) containing 100 μg/ml Spectinomycin (Sigma, St. Louis, Mo.). Plates are incubated overnight at 37° C. Colonies are picked from the plates into 2 ml of 2×LB medium. These liquid cultures are incubated overnight on a roller at 37° C. Plasmid DNA is extracted from these cultures using the Qiagen (Valencia, Calif.) Qiaquik Spin Miniprep kit.

Twelve putative PDE4Bsv1 clones are identified and prepared for a PCR reaction to confirm the presence of the expected PDE4Bsv1 structure. A 25 μl PCR reaction is performed using the Expand High Fidelity PCR System (Roche Applied Science, Indianapolis, Ind.) following manufacturer's instructions to detect the presence of PDE4Bsv1, except that the reaction includes miniprep DNA from the TOPO TA/PDE4Bsv1 cloning reaction as a template. About 10 μl of each 25 μl PCR reaction are run on a 1% agarose gel and the DNA bands generated by the PCR reaction are visualized and photographed on a UV light box to determine which minipreps samples have PCR product of the size predicted for the corresponding PDE4Bsv1 mRNA. Clones having the PDE4Bsv1 structure are identified based upon amplification of an amplicon band of 1,953 base pairs. DNA sequence analysis of the PDE4Bsv1 cloned DNA confirms a polynucleotide sequence representing the deletion of exons 1-7 and presence of exon 1A.

The polynucleotide sequence of PDE4Bsv1 mRNA (SEQ ID NO: 3) lacks a 810 base pair region corresponding to exons 1-7 of the full length coding sequence of the reference PDE4B mRNA (NM_{—}002600) and retains a 102 base pair region deriving from the sequence of intron 7, also known as exon 1A. Conceptual translation of the PDE4Bsv1 mRNA suggests the presence of an alternative start codon in exon 1A, located 48 nucleotides from the 3′ end of exon 1A. The replacement of exons 1-7 with exon 1A and the use of alternative start codon in exon 1A do not alter the protein translation reading frame. Therefore, the PDE4Bsv1 polypeptide possesses a unique N-terminal 16 amino acid region corresponding to exon 1A and is lacking an N-terminal 249 amino acid region corresponding to exons 1-7 of the full length coding sequence of the reference PDE4B mRNA (NM_{—}002600). The unique N-terminal UCR1 and a portion of UCR2 sequence are missing in PDE4Bsv1, but the catalytic domain located in exons 11-16 remains intact. Other PDE4 splice variants which lack UCR1 or UCR1 and a portion of UCR2 demonstrate functional, but altered activity (Bolger et al., 1994, Gene 149:237-244; Sullivan et al., 1998, Biochem. J. 333:693-703; Huston et al., 1997, Biochem. J. 328:549-558).

### Example 3

### Real-time quantitative PCR/TAQman

To determine the relative mRNA abundances of PDE4Bsv1 alternatively spliced isoform to the PDE4B reference transcript (NM_{—}002600), a real-time quantitative PCR assay was used. Materials and methods for quantification of splice variants using real-time PCR, using boundary specific probes are known in the art (Kafert et al., 1999 Anal. Biochem. 269:210-213; Vandenbroucke et al, 2001 Nucleic Acids Res. 29:E68-8; Taveau et al., 2002 Anal. Biochem. 305:227-235).

Reverse Transcription

RNA samples from human fetal brain, heart, lung, and lung carcinoma (ClonTech, Palo Alto, Calif.) were reverse transcribed using the Applied Biosystems (Foster City, Calif.) TAQman reverse transcription kit N808-0234 following manufacturer's instructions. A 50 μl reaction contained:

5 μl 10X RT buffer

11 μl MgCl₂ solution

10 μl dNTP solution

2.5 μl random hexamer primer

1 μl RNAse OUT

3 μl Multiscribe reverse transcriptase

1 μg of RNA

H₂O to a final volume of 50 μl.

To convert RNA to single-stranded cDNA, the reaction mixture was incubated at the following conditions: 25° C. for 10 minutes, 37° C. for 60 minutes, 95° C. for 5 minutes. The cDNA sample was then placed on ice prior to use.

Plasmid Construction and Standard Curve

Plasmids carrying the reference PDE4B sequence and alternatively spliced isoform PDE4Bsv1 were constructed in order to prepare a standard curve. The PDE4Bsv1 cDNA region spanning nucleotides from exon 1A to exon 8 was amplified with exon 1A primer 5′ GCCTGAGGCAAATTATTTGTTATCTGT 3′ (SEQ ID NO:11) and exon 8 primer 5′ GTGTCAGCTCCCGGTTCA 3′ (SEQ ID NO:12) from brain tissue cDNA. The reference PDE4B cDNA region spanning nucleotides from exon 7 to exon 8 was amplified with exon 7 primer 5′ GGTCTGTCAGTGAGATGGCTTCTA 3′ (SEQ ID NO 13) and another exon 8 primer 5′ CCCTGATCGGCTCATCTCTGA 3′ (SEQ ID NO 14) from brain tissue cDNA. The PCR products were cloned into pCR2.1 vector (Invitrogen). The cloning reaction was used to transform TOP10 chemically competent E. coli cells, and plasmid DNA was extracted using the Qiagen (Valencia, Calif.) Qiaquick Spin Miniprep kit. DNA was quantified using a UV spectrometer. Sequence identities of plasmid clones containing the PDE4B reference sequence and alternatively spliced PDE4Bsv1 sequence, which lacks exons 1-7 and contains exon 1A, were verified.

To construct a standard curve with the plasmid clones carrying the PDE4B reference sequence and PDE4Bsv1 sequence, ten-fold serial dilutions of the plasmids were used to obtain a range of five orders of magnitude. Final plasmid concentrations of 100 pg, 10 pg, 1 pg, 0.1 pg, and 0.01 pg were amplified using real-time PCR. Fluorescence emission values were plotted onto a standard curve, permitting quantification of the experimental samples compared to the standard curve.

Real-time PCR

TAQman primers and probes used to quantify the PDE4Bsv1 isoform were designed and synthesized as pre-set mixtures (Applied Biosystems, Foster City, Calif.). The sequences of the TAQman primers and probes used to quantify the PDE4Bsv1 isoform (SEQ ID NOs: 11, 12, and 15) and PDE4B reference form (SEQ ID NOs: 13, 14 and 16) are shown in Table 1. Splice junction specific probes were labeled with the 6-FAM fluorphore at the 5′ end (FAM) and a non-fluorescent quencher at the 3′ end (NFQ). Real-time PCR was performed on human fetal brain, heart, lung, and lung carcinoma cDNA using the TaqMan Universal PCR Master Mix (Applied Biosystems, Foster City, Calif.). The TAQman reaction contained:

| 96-well format | | 384-well format | | |
|---|---|---|---|---|
| 12.5 | μl | 5 | μl | TAQman Universal MasterMix |
| 1.25 | μl | 0.5 | μl | Primer-probe mix |
| 6.25 | μl | 2.5 | μl | H₂O |
| 5 | μl | 2 | μl | cDNA or plasmid DNA. |

TABLE 1
Primers and probes used to quantify PDE4B isoforms.

| | | | |
|---|---|---|---|
| Name | SEQ ID NO: | Sequence | Specificity |

| | | | |
|---|---|---|---|
| PDE4Bsv1 forward primer | SEQ ID NO:11 | GCCTGAGGCAAATTATTTGTTATCTGT | PDE4Bsv1 |
| PDE4Bsv1 reverse primer | SEQ ID NO:12 | GTGTCAGCTCCCGGTTCA | PDE4Bsv1 |
| PDE4Bsv1 probe | SEQ ID NO:15 | FAM-CATCAAGTTCAAAAGAATGC-NFQ | PDE4Bsv1 |
| PDE4B reference forward | SEQ ID NO:13 | GGTCTGTCAGTGAGATGGCTTCTA | PDE4B |
| primer | | | reference |
| PDE4B reference reverse | SEQ ID NO:14 | CCCTGATCGGCTCATCTCTGA | PDE4B |
| primer | | | reference |
| PDE4B reference probe | SEQ ID NO:16 | FAM-ACAAGTTCAAAAGAATGCTGAA-NFQ | PDE4B |
| | | | reference |

The TAQman reactions were performed on an ABI Prism 7900HT Sequence Detection System (Applied Biosystems, Foster City, Calif.). The thermocycling conditions were 50° C. for 2 minutes, 95° C. for 10 minutes, and 40 cycles of 95° C. for 15 seconds and 60° C. for 1 minute. Data analysis of the fluorescence emission was performed by the Sequence Detector Software (SDS) (Applied Biosystems, Foster City, Calif.). Briefly, an amplification plot was generated for each sample, which showed cycle number on the x axis vs. ΔRₙ on they axis. Rₙ is the fluorescence emission intensity of the reporter dye normalized to a passive reference, and ΔRₙ is the Rₙ value of the reaction minus the Rₙ, of an un-reacted sample. A threshold cycle (C_{T}) value, the cycle at which a statistically significant increase in ΔRₙ is first detected, was calculated from the amplification plot. The threshold was automatically calculated by the SDS as the 10-fold standard deviation of the Rₙ, in the first 15 cycles. The obtained C_{T} values were exported Microsoft Excel for analysis as recommended by the manufacturer (Applied Biosystems, Foster City, Calif.). Standard curve plots showing the log₁₀ [input cDNA] vs. C_{T} values were constructed. Referring to the standard curve, C_{T} values for the experimental samples were then used to calculate the input amount of the PDE4B isoform cDNA. The most highly expressed isoform, in this case the reference form of PDE4B from lung carcinoma tissue, was assigned the arbitrary value of 100%, and other isoforms from other tissues were presented as percentages the most highly expressed isoform. Quantitative analysis of the real-time PCR data indicated that the reference PDE4B is most abundant in lung carcinoma tissue compared to other human tissues: 67.5% in fetal brain, 15.2% in heart, and 11.7% in lung, (normalized to level of reference PDE4B in lung carcinoma tissue=100%). Quantitative analysis of the real-time PCR data indicated that the PDE4Bsv1 isoform was most abundant in fetal brain tissue, but was less abundant than the reference PDE4B in other tissues examined: 0% in heart, 0% in lung, and 2.6% in lung carcinoma (normalized to level of reference PDE4B in lung carcinoma=100%). These results demonstrate that the PDE4Bsv1 isoform is most abundant in fetal brain over lung carcinoma, heart, and lung and is more abundant than the reference PDE4B in fetal brain.

### Example 4

### Cloning of PDE4Bsv1

Method 2:

Clones having a nucleotide sequence comprising the splice variant identified in Example 1 (hereafter referred to as PDE4Bsv1) were isolated using PDE4B1, the long reference form (Genbank Accession Number L20966; SEQ ID NO: 17) as a PCR template to incorporate the novel N-terminal region of PDE4Bsv1 into the new construct. A 5′ “forward” PDE4Bsv1 primer was designed to contain a Not I restriction enzyme site, the sequence encoding the novel 16 N-terminal amino acids of PDE4sv 1, and sequence encoding 6 amino acids of PDE4Bsv1 at positions 249-254. The 3′ “reverse” PDE4Bsv1 primer was designed to contain sequences encoding amino acids from the C-terminal region of PDE4Bsv1, a stop codon, and a Kpn I restriction enzyme site. A FLAG tag (DYKDDDDK, SEQ ID NO: 18) may also be incorporated into the 3′ “reverse” PDE4Bsv1 primer before the stop codon.

A PCR reaction using Taq DNA polymerase, PDE4B1 DNA template, and the above-described primers generated a PCR product of approximately 1,558 base pairs. The amplicon was then purified using PCR Purification Kit (Qiagen, Crawley, UK) and then digested with Not I and Kpn 1. The resulting fragment was then ligated (Rapid DNA Ligation Kit, Roche Diagnostic GmbH, Mannheim, Germany) into the MCS of pcDNA3.1 vector (Invitrogen, Paisley, UK) to generate the PDE4Bsv1-pcDNA3 construct.

### Example 5

### Transient Expression of PDE4Bsv1

The PDE4Bsv1-pcDNA3 construct from cloning Method 2 of Example 4 was introduced into COS-7 SV40-transformed monkey kidney cell line (ATCC #CRL-1651). The COS-7 cells were cultured in Dulbecco's modified Eagle's medium (DMEM) supplemented with 0.1% penicillin/streptomycin (10,000 units/ml), glutamine (2 mM), and 10% fetal calf serum at 37° C. and 5% CO₂. The COS-7 cells were transfected with the PDE4Bsv1-pcDNA3 construct using DEAE-dextran as previously described (Huston et al., 1997, Biochem. J. 328:549-558; Huston et al., 1996, J. Biol. Chem. 271:31334-31344; Rena et al., 2001, Mol. Pharmacol. 59:996-1011; Wallace et al., 2005, Mol. Pharmacol. 67:1920-1934). The PDE4Bsv1-pcDNA3 construct (10 μg) was mixed and incubated with 200 μl of DEAE-dextran/PBS (10 mg/ml) for 15 minutes to produce a “DNA-dextran” mix. The COS-7 cells were grown in 100 mm Petri dishes to 70% confluence, and the culture medium was then removed. 10 ml of fresh DMEM containing 0.1 mM chloroquine and the DNA-dextran mix (450 μl) were added to the Petri dish with the COS-7 cells. The cells were then incubated for 4 hours at 37° C. After the incubation, the culture medium was aspirated and the COS-7 cells were shocked for 2 minutes with 10% DMSO in PBS. After two PBS washes, the COS-7 cells were incubated in normal culture medium for 2 days.

For determination of PDE enzyme activity, the transfected COS-7 cells were homogenized in KHEM buffer (50 mM KCl, 50 mM HEPES/KOH, 10 mM EGTA, 1.92 mM MgCl₂, 1 mM dithiothreitol, final pH 7.2) containing final concentrations of the following “complete” protease inhibitors: PMSF (40 μg/ml); benzamine (156 μg/ml); aprotonin (1 μg/ml); leupeptin (1 μg/ml); pepstatin A (1 μg/ml); antipain (1 μg/ml). As previously described (Huston et al., 1997, supra; Huston et al., 1996, supra), in transfected cells treated in this manner, >98% of the total PDE activity is due to the recombinant PDE4 isoform. In some instances, the transfected COS-7 cells were plated onto 6-well tissue culture plates for use in activity assays and then serum-starved overnight prior to treatment with substrates.

### Example 6

### PDE Activity Assay

PDE activity was determined using a modified two-step radioassay procedure of Thompson and Appleman (1971, Biochemistry 10:311-316) as previously described (Marchmont and Houslay, 1980, Biochem. J. 187:381-392; Huston et al., 1996, supra; Rena et al., 2001, supra; Sullivan et al., 1998, Biochem. J. 333:693-703). All assays were conducted at 30° C., and in all experiments, a freshly prepared slurry of Dowex/water/ethanol (1:1:1, by volume) was used for determination of activities. Initial rates were taken from linear time-courses of activity.

Total PDE4 activity in COS-7 transfected cells was determined at a cAMP substrate concentration of 1 μM and defined as that amount of PDE activity that could be inhibited by the addition of 10 μM rolipram. This is a concentration at which rolipram serves as a PDE4-selective inhibitor and can completely inhibit PDE4 activity (Houslay et al., 1998, Adv. Pharmacol. 44: 225-242). Over 97% of the total cAMP PDE activity was inhibited by 1 μM cAMP rolipram as substrate. In assays with 1 μM cAMP substrate, COS-7 cells transfected with the PDE4Bsv1 construct had PDE activity of 24 nmol cAMP hydrolyzed/min/mg cell protein while COS-7 cells mock transfected with vector only had an activity of 4-6 pmol cAMP hydrolyzed/min/mg cell protein (n=3). Thus, in PDE4Bsv1 transfected COS-7 cells, the PDE4Bsv1 construct comprised >98% of the total COS cell PDE activity.

For the determination of kinetic parameters, the PDE assays were conducted with a range of cAMP concentrations. Analysis of PDE4Bsv1 showed that it had a Kₘ of 5.8±0.4 μM (n=3). For Vₘₐₓ determinations, equal amounts of PDE4Bsv1 and PDE4B2 (NM_{—}001037339; SEQ ID NO: 19) from transfected COS-7 cell lysates were compared, showing that the Vₘₐₓ of PDE4Bsv1 was 18±3% of PDE4B2.

The sensitivity of PDE4Bsv1 to rolipram and to another PDE4 selective inhibitor, cilomilast (GlaxoSmithKline), was determined. The IC₅₀ values for rolipram and cilomilast were 380+63 nM (n=4) and 114+17 nM (n=3), respectively. Rolipram binds to the catalytic site of PDE4B, thus providing competitive inhibition. Using the Cheng-Prussof equation (Kᵢ=IC₅₀/(I+(S/Kₘ))), Kᵢ values for inhibition of PDE4Bsv1 by rolipram and cilomilast were determined to be 324 and 97 nM, respectively.

### Example 7

### Intracellular Distribution of PDE4Bsv1

COS-7 cells were transfected with the PDE4Bsv1-pcDNA3 expression construct as described in Example 5. The distribution of PDE4Bsv1 between low speed membrane (P1), high speed membrane (P2), and high speed supernatant (S2) cytosolic fractions was assessed immunologically by Western blot analysis. Transfected COS-7 cells were disrupted as described (Bolger et al., 1996, J. Biol. Chem. 271:1065-1071; Huston et al., 1997, Biochem. J. 328:549-558; McPhee et al., 1995, Biochem. J. 310:965-974; Shakur et al., 1993, Biochem. J. 292:677-686; Shakur et al., 1995, Biochem. J. 306:801-809). Cells were homogenized in KHEM buffer described in Example 5. Pellet fractions were also resuspended in this mixture. A low-speed P1 pellet (1000 gₐᵥ for 10 minutes) and a high-speed P2 pellet (100,000 gₐᵥ for 60 minutes), as well as a high-speed supernatant (S2) were generated. The homogenization procedures was complete in that there was no detectable latent lactate dehydrogenase activity present in the P1 pellet, indicating an absence of cytosolic proteins. Equal volumes of samples were assayed such that detection indicated relative distribution among these three sub-cellular fractions.

Sub-cellular fractions were resuspended in SDS buffer and were run on acrylamide gels (4-12%) at 100 V/gel for 1-2 hours with cooling and then transferred to nitrocellulose membranes before immunoblotting using specific polyclonal antisera. The polyclonal antisera was raised against the extreme C-terminal region that is unique to the PDE4B sub-family and is found in all known active PDE4B isoforms, as described in Huston et al., 1997, supra. Labeled bands were then identified using anti-rabbit peroxidase-linked IgG, and the Amersham Biosciences ECL Western blotting system was used as a visualization protocol for detection and quantification. Immunblot detection of the recombinant PDE4Bsv1 from the transfected COS-7 sub-cellular fractions showed a 58 kDa band, which is in agreement with the predicted size of 57.7 kDa (data not shown). Distribution of PDE4Bsv1 among the sub-cellular fractions, as detected by immunoblot, is shown in Table 2. This analysis showed that PDE4Bsv1 was found predominantly in the S2 high speed supernatant, cytosolic fraction. However, about 30% of the total PDE4Bsv1 was also associated with the membrane fractions.

Determination of PDE4 activity among the sub-cellular fractions was also determined by using 1 μM cAMP as substrate as described in Example 6. Sub-cellular distribution of PDE4Bsv1activity was in agreement with its distribution (see Table 2).

TABLE 2
Intracellular distribution of PDE4Bsv1

| | Immunoreactivity | |
|---|---|---|
| Sub-cellular fraction | distribution (%) | Activity distribution (%) |
| P1 | 13 ± 2 | 12 ± 2 |
| P2 | 17 ± 2 | 15 ± 2 |
| S2 | 70 ± 7 | 73 ± 7 |

All patents, patent publications, and other published references mentioned herein are hereby incorporated by reference in their entireties as if each had been individually and specifically incorporated by reference herein. While preferred illustrative embodiments of the present invention are shown and described, one skilled in the art will appreciate that the present invention can be practiced by other than the described embodiments, which are presented for purposes of illustration only and not by way of limitation. Various modifications may be made to the embodiments described herein without departing from the spirit and scope of the present invention. The present invention is limited only by the claims that follow.

What is claimed is:

1. A purified human nucleic acid comprising SEQ ID NO: 3, or the complement thereof.

2. The purified nucleic acid of claim 1, wherein said nucleic acid comprises a sequence encoding SEQ ID NO: 4.

3. The purified nucleic acid of claim 1, wherein said nucleic acid encodes a polypeptide consisting of SEQ ID NO: 4.

4. A purified polypeptide comprising SEQ ID NO: 4.

5. The polypeptide of claim 4, wherein said polypeptide consists of SEQ ID NO: 4.

6. An expression vector comprising a nucleotide sequence encoding SEQ ID NO: 4, wherein said nucleotide sequence is transcriptionally coupled to an exogenous promoter.

7. The expression vector of claim 6, wherein said nucleotide sequence encodes a polypeptide consisting of SEQ ID NO: 4.

8. The expression vector of claim 4, wherein said nucleotide sequence comprises SEQ ID NO: 3.

9. The expression vector of claim 6, wherein said nucleotide sequence consists of SEQ ID NO: 3.

10. A method of screening for compounds able to bind selectively to PDE4Bsv1comprising the steps of:
(a) providing a PDE4Bsv1 polypeptide comprising SEQ ID NO: 4;
(b) providing one or more PDE isoform polypeptides that are not PDE4Bsv1;
(c) contacting said PDE4Bsv1 polypeptide and said PDE isoform polypeptide that is not PDE4Bsv1 with a test preparation comprising one or more compounds; and
(d) determining the binding of said test preparation to said PDE4Bsv1polypeptide and to said PDE isoform polypeptide that is not PDE4Bsv1, wherein a test preparation which binds to said PDE4Bsv1 polypeptide, but does not bind to said PDE isoform polypeptide that is not PDE4Bsv1, contains a compound that selectively binds said PDE4Bsv1 polypeptide.

11. The method of claim 10, wherein said PDE4Bsv1 polypeptide is obtained by expression of said polypeptide from an expression vector comprising a polynucleotide encoding SEQ ID NO: 4.

12. The method of claim 11, wherein said polypeptide consists of SEQ ID NO: 4.

13. A method for screening for a compound able to bind to or interact with a PDE4Bsv1 protein or a fragment thereof comprising the steps of:
(a) expressing a PDE4Bsv1 polypeptide comprising SEQ ID NO: 4 or fragment thereof from a recombinant nucleic acid;
(b) providing to said polypeptide a labeled PDE ligand that binds to said polypeptide and a test preparation comprising one or more compounds; and
(c) measuring the effect of said test preparation on binding of said labeled PDE ligand to said polypeptide, wherein a test preparation that alters the binding of said labeled PDE ligand to said polypeptide contains a compound that binds to or interacts with said polypeptide.

14. The method of claim 13, wherein said steps (b) and (c) are performed in vitro.

15. The method of claim 13, wherein said steps (a), (b) and (c) are performed using a whole cell.

16. The method of claim 13, wherein said polypeptide is expressed from an expression vector.

17. The method of claim 13, wherein said PDE4Bsv1 ligand is a PDE inhibitor.

18. The method of claim 16, wherein said expression vector comprises SEQ ID NO: 3 or a fragment of SEQ ID NO: 3.

19. The method of claim 16, wherein said polypeptide comprises SEQ ID NO: 4 or a fragment of SEQ ID NO: 4.

20. A method of screening for a compound that modulates activity of PDE4Bsv1comprising:
(a) expressing a recombinant nucleic acid encoding PDE4Bsv1 comprising SEQ ID NO: 4 in a cell;
(b) contacting said cell or a cell extract thereof with a test preparation comprising one or more test compounds; and
(c) measuring the effect of said test preparation on enzyme activity.


SEQUENCE LISTING (19 sequences)

SEQ ID NO: 1 - DNA, 40 bp, Homo sapiens
tatcttggggttacatcaagttcaaaagaatgctgaaccg

SEQ ID NO: 2 - DNA, 102 bp, Homo sapiens
actgtgaattctttcaaagggatttgtggattgtggcaaggagagcattccaaaatgcctgaggcaaattatttgttatctgtatcttggggttacatcaag

SEQ ID NO: 3 - DNA, 2403 bp, Homo sapiens
actgtgaattctttcaaagggatttgtggattgtggcaaggagagcattccaaaatgcctgaggcaaattatttgttatctgtatcttggggttacatcaagttcaaaagaatgctgaaccgggagctgacacacctctcagagatgagccgatcagggaaccaggtgtctgaatacatttcaaatactttcttagacaagcagaatgatgtggagatcccatctcctacccagaaagacagggagaaaaagaaaaagcagcagctcatgacccagataagtggagtgaagaaattaatgcatagttcaagcctaaacaatacaagcatctcacgctttggagtcaacactgaaaatgaagatcacctggccaaggagctggaagacctgaacaaatggggtcttaacatctttaatgtggctggatattctcacaatagacccctaacatgcatcatgtatgctatattccaggaaagagacctcctaaagacattcagaatctcatctgacacatttataacctacatgatgactttagaagaccattaccattctgacgtggcatatcacaacagcctgcacgctgctgatgtagcccagtcgacccatgttctcctttctacaccagcattagacgctgtcttcacagatttggagatcctggctgccatttttgcagctgccatccatgacgttgatcatcctggagtctccaatcagtttctcatcaacacaaattcagaacttgctttgatgtataatgatgaatctgtgttggaaaatcatcaccttgctgtgggtttcaaactgctgcaagaagaacactgtgacatcttcatgaatctcaccaagaagcagcgtcagacactcaggaagatggttattgacatggtgttagcaactgatatgtctaaacatatgagcctgctggcagacctgaagacaatggtagaaacgaagaaagttacaagttcaggcgttcttctcctagacaactataccgatcgcattcaggtccttcgcaacatggtacactgtgcagacctgagcaaccccaccaagtccttggaattgtatcggcaatggacagaccgcatcatggaggaatttttccagcagggagacaaagagcgggagaggggaatggaaattagcccaatgtgtgataaacacacagcttctgtggaaaaatcccaggttggtttcatcgactacattgtccatccattgtgggagacatgggcagatttggtacagcctgatgctcaggacattctcgataccttagaagataacaggaactggtatcagagcatgatacctcaaagtccctcaccaccactggacgagcagaacagggactgccagggtctgatggagaagtttcagtttgaactgactctcgatgaggaagattctgaaggacctgagaaggagggagagggacacagctatttcagcagcacaaagacgctttgtgtgattgatccagaaaacagagattccctgggagagactgacatagacattgcaacagaagacaagtcccccgtggatacataatccccctctccctgtggagatgaacattctatccttgatgagcatgccagctatgtggtagggccagcccaccatgggggccaagacctgcacaggacaagggccacctggcctttcagttacttgagtttggagtcagaaagcaagaccaggaagcaaatagcagctcaggaaatcccacggttgacttgccttgatggcaagcttggtggagagggctgaagctgttgctgggggccgattctgatcaagacacatggcttgaaaatggaagacacaaaactgagagatcattctgcactaagtttcgggaacttatccccgacagtgactgaactcactgactaataacttcatttatgaatcttctcacttgtccctttgtctgccaacctgtgtgccttttttgtaaaacattttcatgtctttaaaatgcctgttgaatacctggagtttagtatcaacttctacacagataagctttcaaagttgacaaacttttttgactctttctggaaaagggaaagaaaatagtcttccttctttcttgggcaatatccttcactttactacagttacttttgcaaacagacagaaaggatacacttctaaccacattttacttccttcccctgttgtccagtccaactccacagtcactcttaaaacttctctctgtttgcctgcctccaacagtacttttaactttttgctgtaaacagaataaaattgaacaaattagggggtagaaaggagcagtggtgtcgttcaccgtgagagtctgcatagaactcagcagtgtgccctgctgtgtcttggaccctgcaatgcggccgc

SEQ ID NO: 4 - protein, 503 aa, Homo sapiens
MPEANYLLSVSWGYIKFKRMLNRELTHLSEMSRSGNQVSEYISNTFLDKQNDVEIPSPTQKDREKKKKQQLMTQISGVKKLMHSSSLNNTSISRFGVNTENEDHLAKELEDLNKWGLNIFNVAGYSHNRPLTCIMYAIFQERDLLKTFRISSDTFITYMMTLEDHYHSDVAYHNSLHAADVAQSTHVLLSTPALDAVFTDLEILAAIFAAAIHDVDHPGVSNQFLINTNSELALMYNDESVLENHHLAVGFKLLQEEHCDIFMNLTKKQRQTLRKMVIDMVLATDMSKHMSLLADLKTMVETKKVTSSGVLLLDNYTDRIQVLRNMVHCADLSNPTKSLELYRQWTDRIMEEFFQQGDKERERGMEISPMCDKHTASVEKSQVGFIDYIVHPLWETWADLVQPDAQDILDTLEDNRNWYQSMIPQSPSPPLDEQNRDCQGLMEKFQFELTLDEEDSEGPEKEGEGHSYFSSTKTLCVIDPENRDSLGETDIDIATEDKSPVDT

SEQ ID NO: 5 - DNA, 20 bp, Homo sapiens
ttacatcaagttcaaaagaa

SEQ ID NO: 6 - protein, 10 aa, Homo sapiens
WGYIKFKRML

SEQ ID NO: 7 - DNA, 28 bp, Artificial Sequence
  Oligonucleotide primer
actgtgaattctttcaaagggatttgtg

SEQ ID NO: 8 - DNA, 28 bp, Artificial Sequence
  Oligonucleotide primer
ggtctattgtgagaatatccagccacat

SEQ ID NO: 9 - DNA, 31 bp, Artificial Sequence
  Oligonucleotide primer
agatggctgtgtttcctagtctggcaactcc

SEQ ID NO: 10 - DNA, 33 bp, Artificial Sequence
  Oligonucleotide primer
ttatgtatccacgggggacttgtcttctgttgc

SEQ ID NO: 11 - DNA, 27 bp, Artificial Sequence
  Oligonucleotide primer
gcctgaggcaaattatttgttatctgt

SEQ ID NO: 12 - DNA, 18 bp, Artificial Sequence
  Oligonucleotide primer
gtgtcagctcccggttca

SEQ ID NO: 13 - DNA, 24 bp, Artificial Sequence
  Oligonucleotide primer
ggtctgtcagtgagatggcttcta

SEQ ID NO: 14 - DNA, 21 bp, Artificial Sequence
  Oligonucleotide primer
ccctgatcggctcatctctga

SEQ ID NO: 15 - DNA, 20 bp, Artificial Sequence
  Oligonucleotide probe
catcaagttcaaaagaatgc

SEQ ID NO: 16 - DNA, 22 bp, Artificial Sequence
  Oligonucleotide probe
acaagttcaaaagaatgctgaa

SEQ ID NO: 17 - DNA, 3186 bp, Homo sapiens
gcggccgcggcggtgcagcagaggcgcctcgggcaggaggagggcggcttctgcgagggcagcctgaggtattaaaaagtgtcagcaaactgcattgaataacagacatcctaagaggggatattttccacctctataatgaagaaaagcaggagtgtgatgacggtgatggctgatgataatgttaaagattattttgaatgtagcttgagtaaatcctacagttcttccagtaacacacttgggatcgacctctggagagggagaaggtgttgctcaggaaacttacagttaccaccactgtctcaaagacagagtgaaagggcaaggactcctgagggagatggtatttccaggccgaccacactgcctttgacaacgcttccaagcattgctattacaactgtaagccaggagtgctttgatgtggaaaatggcccttccccaggtcggagtccactggatccccaggccagctcttccgctgggctggtacttcacgccacctttcctgggcacagccagcgcagagagtcatttctctacagatcagacagcgactatgacttgtcaccaaaggcgatgtcgagaaactcttctcttccaagcgagcaacacggcgatgacttgattgtaactccttttgcccaggtccttgccagcttgcgaagtgtgagaaacaacttcactatactgacaaaccttcatggtacatctaacaagaggtccccagctgctagtcagcctcctgtctccagagtcaacccacaagaagaatcttatcaaaaattagcaatggaaacgctggaggaattagactggtgtttagaccagctagagaccatacagacctaccggtctgtcagtgagatggcttctaacaagttcaaaagaatgctgaaccgggagctgacacacctctcagagatgagccgatcagggaaccaggtgtctgaatacatttcaaatactttcttagacaagcagaatgatgtggagatcccatctcctacccagaaagacagggagaaaaagaaaaagcagcagctcatgacccagataagtggagtgaagaaattaatgcatagttcaagcctaaacaatacaagcatctcacgctttggagtcaacactgaaaatgaagatcacctggccaaggagctggaagacctgaacaaatggggtcttaacatctttaatgtggctggatattctcacaatagacccctaacatgcatcatgtatgctatattccaggaaagagacctcctaaagacattcagaatctcatctgacacatttataacctacatgatgactttagaagaccattaccattctgacgtggcatatcacaacagcctgcacgctgctgatgtagcccagtcgacccatgttctcctttctacaccagcattagacgctgtcttcacagatttggagatcctggctgccatttttgcagctgccatccatgacgttgatcatcctggagtctccaatcagtttctcatcaacacaaattcagaacttgctttgatgtataatgatgaatctgtgttggaaaatcatcaccttgctgtgggtttcaaactgctgcaagaagaacactgtgacatcttcatgaatctcaccaagaagcagcgtcagacactcaggaagatggttattgacatggtgttagcaactgatatgtctaaacatatgagcctgctggcagacctgaagacaatggtagaaacgaagaaagttacaagttcaggcgttcttctcctagacaactataccgatcgcattcaggtccttcgcaacatggtacactgtgcagacctgagcaaccccaccaagtccttggaattgtatcggcaatggacagaccgcatcatggaggaatttttccagcagggagacaaagagcgggagaggggaatggaaattagcccaatgtgtgataaacacacagcttctgtggaaaaatcccaggttggtttcatcgactacattgtccatccattgtgggagacatgggcagatttggtacagcctgatgctcaggacattctcgataccttagaagataacaggaactggtatcagagcatgatacctcaaagtccctcaccaccactggacgagcagaacagggactgccagggtctgatggagaagtttcagtttgaactgactctcgatgaggaagattctgaaggacctgagaaggagggagagggacacagctatttcagcagcacaaagacgctttgtgtgattgatccagaaaacagagattccctgggagagactgacatagacattgcaacagaagacaagtcccccgtggatacataatccccctctccctgtggagatgaacattctatccttgatgagcatgccagctatgtggtagggccagcccaccatgggggccaagacctgcacaggacaagggccacctggcctttcagttacttgagtttggagtcagaaagcaagaccaggaagcaaatagcagctcaggaaatcccacggttgacttgccttgatggcaagcttggtggagagggctgaagctgttgctgggggccgattctgatcaagacacatggcttgaaaatggaagacacaaaactgagagatcattctgcactaagtttcgggaacttatccccgacagtgactgaactcactgactaataacttcatttatgaatcttctcacttgtccctttgtctgccaacctgtgtgccttttttgtaaaacattttcatgtctttaaaatgcctgttgaatacctggagtttagtatcaacttctacacagataagctttcaaagttgacaaacttttttgactctttctggaaaagggaaagaaaatagtcttccttctttcttgggcaatatccttcactttactacagttacttttgcaaacagacagaaaggatacacttctaaccacattttacttccttcccctgttgtccagtccaactccacagtcactcttaaaacttctctctgtttgcctgcctccaacagtacttttaactttttgctgtaaacagaataaaattgaacaaattagggggtagaaaggagcagtggtgtcgttcaccgtgagagtctgcatagaactcagcagtgtgccctgctgtgtcttggaccctgcaatgcggccgc

SEQ ID NO: 18 - protein, 8 aa, Artificial Sequence
  FLAG peptide tag
DYKDDDDK

SEQ ID NO: 19 - DNA, 3882 bp, Homo sapiens
cacataccctaaagaaccctgggatgactaaggcagagagagtctgagaaaactctttggtgcttctgcctttagttttaggacacatttatgcagatgagcttataagagaccgttccctccgccttcttcctcagaggaagtttcttggtagatcaccgacacctcatccaggcggggggttggggggaaacttggcaccagccatcccaggcagagcaccactgtgatttgttctcctggtggagagagctggaaggaaggagccagcgtgcaaataatgaaggagcacgggggcaccttcagtagcaccggaatcagcggtggtagcggtgactctgctatggacagcctgcagccgctccagcctaactacatgcctgtgtgtttgtttgcagaagaatcttatcaaaaattagcaatggaaacgctggaggaattagactggtgtttagaccagctagagaccatacagacctaccggtctgtcagtgagatggcttctaacaagttcaaaagaatgctgaaccgggagctgacacacctctcagagatgagccgatcagggaaccaggtgtctgaatacatttcaaatactttcttagacaagcagaatgatgtggagatcccatctcctacccagaaagacagggagaaaaagaaaaagcagcagctcatgacccagataagtggagtgaagaaattaatgcatagttcaagcctaaacaatacaagcatctcacgctttggagtcaacactgaaaatgaagatcacctggccaaggagctggaagacctgaacaaatggggtcttaacatctttaatgtggctggatattctcacaatagacccctaacatgcatcatgtatgctatattccaggaaagagacctcctaaagacattcagaatctcatctgacacatttataacctacatgatgactttagaagaccattaccattctgacgtggcatatcacaacagcctgcacgctgctgatgtagcccagtcgacccatgttctcctttctacaccagcattagacgctgtcttcacagatttggagatcctggctgccatttttgcagctgccatccatgacgttgatcatcctggagtctccaatcagtttctcatcaacacaaattcagaacttgctttgatgtataatgatgaatctgtgttggaaaatcatcaccttgctgtgggtttcaaactgctgcaagaagaacactgtgacatcttcatgaatctcaccaagaagcagcgtcagacactcaggaagatggttattgacatggtgttagcaactgatatgtctaaacatatgagcctgctggcagacctgaagacaatggtagaaacgaagaaagttacaagttcaggcgttcttctcctagacaactataccgatcgcattcaggtccttcgcaacatggtacactgtgcagacctgagcaaccccaccaagtccttggaattgtatcggcaatggacagaccgcatcatggaggaatttttccagcagggagacaaagagcgggagaggggaatggaaattagcccaatgtgtgataaacacacagcttctgtggaaaaatcccaggttggtttcatcgactacattgtccatccattgtgggagacatgggcagatttggtacagcctgatgctcaggacattctcgataccttagaagataacaggaactggtatcagagcatgatacctcaaagtccctcaccaccactggacgagcagaacagggactgccagggtctgatggagaagtttcagtttgaactgactctcgatgaggaagattctgaaggacctgagaaggagggagagggacacagctatttcagcagcacaaagacgctttgtgtgattgatccagaaaacagagattccctgggagagactgacatagacattgcaacagaagacaagtcccccgtggatacataatccccctctccctgtggagatgaacattctatccttgatgagcatgccagctatgtggtagggccagcccaccatgggggccaagacctgcacaggacaagggccacctggcctttcagttacttgagtttggagtcagaaagcaagaccaggaagcaaatagcagctcaggaaatcccacggttgacttgccttgatggcaagcttggtggagagggctgaagctgttgctgggggccgattctgatcaagacacatggcttgaaaatggaagacacaaaactgagagatcattctgcactaagtttcgggaacttatccccgacagtgactgaactcactgactaataacttcatttatgaatcttctcacttgtccctttgtctgccaacctgtgtgccttttttgtaaaacattttcatgtctttaaaatgcctgttgaatacctggagtttagtatcaacttctacacagataagctttcaaagttgacaaacttttttgactctttctggaaaagggaaagaaaatagtcttccttctttcttgggcaatatccttcactttactacagttacttttgcaaacagacagaaaggatacacttctaaccacattttacttccttcccctgttgtccagtccaactccacagtcactcttaaaacttctctctgtttgcctgcctccaacagtacttttaactttttgctgtaaacagaataaaattgaacaaattagggggtagaaaggagcagtggtgtcgttcaccgtgagagtctgcatagaactcagcagtgtgccctgctgtgtcttggaccctgccccccacaggagttgtacagtccctggccctgttccctacctcctctcttcaccccgttaggctgttttcaatgtaatgctgccgtccttctcttgcactgccttctgcgctaacacctccattcctgtttataaccgtgtatttattacttaatgtatataatgtaatgttttgtaagttattaatttatatatctaacattgcctgccaatggtggtgttaaatttgtgtagaaaactctgcctaagagttacgactttttcttgtaatgttttgtattgtgtattatataacccaaacgtcacttagtagagacatatggcccccttggcagagaggacaggggtgggcttttgttcaaagggtctgccctttccctgcctgagttgctacttctgcacaacccctttatgaaccagttttggaaacaatattctcacattagatactaaatggtttatactgagcttttacttttgtatagcttgataggggcagggggcaatgggatgtagtttttacccaggttctatccaaatctatgtgggcatgagttgggttataactggatcctactatcattgtggctttggttcaaaaggaaacactacatttgctcacagatgattcttctgaatgctcccgaactactgactttgaagaggtagcctcctgcctgccattaagcaggaatgtcatgttccagttcattacaaaagaaaacaataaaacaatgtgaatttttataataaaatgtgaactgatgtagcaaattacgcaaatgtgaagcctcttctgataacacttgttaggcctcttactgatgtcagtttcagtttgtaaaatatgtttcatgctttcagttcagcattgtgactcagtaattacagaaaatggcacaaatgtgcatgaccaatgtatgtctatgaacactgcattgtttcaggtggacattttatcattttcaaatgtttctcacaatgtatgttatagtattattattatatattgtgttcaaatgcattctaaagagacttttatatgaggtgaataaagaaaagcatgattagattaaaaaaa